(19) United States
(12) Patent Application Publication
(10) Pub. No.: US 2003/0211545 A1
(43) Pub. Date: Nov. 13, 2003

(54) Treatment of neuropsychiatric disease with protease and neurominidase inhibitors

(76) Inventors: Marquis P. Vawter, Laguna Lagalle, CA (US); William J. Freed, Bowie, MD (US)

Correspondence Address: NEEDLE & ROSENBERG, P.C., SUITE 1000, 999 PEACHTREE STREET, ATLANTA, GA, 30309-3915, US

(21) Appl. No.: 10/182,162

(22) PCT Filed: Jan. 25, 2001
(86) PCT No.: PCT/US01/02417

(51) Int. Cl. G01N 33/53; G01N 33/567; A61K 38/08; A61K 31/551

(52) U.S. Cl. 435/7.2; 514/220; 514/17

(57) ABSTRACT

The present invention provides a method of treating a neuropsychiatric disease characterized by an abnormally elevated level of a fragment of an isoform of a neural cell adhesion molecule, N-CAM, in the brain or cerebrospinal fluid of an affected human subject, comprising administering a therapeutically effective amount of at least one compound selected from the group consisting of protease inhibitors and neuraminidase inhibitors, whereby administering the compound to the subject treats the human subject. The present invention further provides a method of monitoring the efficacy of treatment with the method of the present invention. Moreover, the present invention provides a method of screening for compounds effective in treating neuropsychiatric disease associated with an abnormally elevated level of a fragment of a neural cell adhesion molecule in the cerebrospinal fluid of an affected human subject. Further provided are fragments of an isoform of N-CAM in the cerebrospinal fluid of human subjects.

### BACKGROUND OF THE INVENTION

1. Field of the Invention

The present invention relates to the field of therapy of neuropsychiatric diseases in human subjects. In particular, the present invention relates to a method of treating neuropsychiatric diseases associated with an abnormally elevated level of a fragment of a neural cell adhesion molecule (N-CAM) in brain or cerebrospinal fluid, comprising administering a protease inhibitor, a neuraminidase inhibitor or a combination of both with or without a neuroleptic medication.

2. Background Art

Historically, neuropsychiatric research has sought answers to the pathogenesis and progression of Schizophrenia, Bipolar Disorder I, Bipolar Disorder I with Psychotic Features, Bipolar Disorder II, Bipolar Disorder II with Psychotic Features, Psychotic Disorder Not Otherwise Specified, Schizophreniform Disorder, Schizoaffective Disorder, Unipolar Disorder, Unipolar Disorder with Psychotic Features, Substance Induced Psychotic Disorder, Schizotypal Personality Disorder and Mood Disorder with Psychotic Features, diseases which share many similar features. Many mechanisms have been described, e.g. obstetric complications such as hypoxia, viral infections, immune disorders, and malnutrition which can later be associated with subgroups of patients that are susceptible to developing neuropsychiatric diseases.

Schizophrenia is a life-long illness with multiple features that are defined by DSM-IV criteria and with additional features accepted by those skilled in the art: increased ventricle size of the brain, thinning of the cortical grey matter, and cognitive decline. These 3 clinical observations have been made repeatedly by comparing groups of patients with schizophrenia and controls, and although there are group differences, there are no definitive tests for schizophrenia. A subjective diagnosis for these disorders is made by a trained clinician following criteria set forth in the DSM-IV which then allows for a heterogeneous group of disorders with overlap since several criteria can substitute for one another. Thus, the multiple manifestations of schizophrenia overlap with other conditions defined in the DSM-IV. Nevertheless, the cardinal symptoms of schizophrenia can be generally grouped into three groups; positive, or expressive symptomatology; negative, or deficit, symptomatology; and disorganized symptomatology further defined in the DSM-IV.

Because many patients with Schizophrenia, Bipolar Disorder I, Bipolar Disorder I with Psychotic Features, Bipolar Disorder II, Bipolar Disorder II with Psychotic Features, Psychotic Disorder Not Otherwise Specified, Schizophreniform Disorder, Schizoaffective Disorder, Unipolar Disorder, Unipolar Disorder with Psychotic Features, Substance Induced Psychotic Disorder, Schizotypal Personality Disorder and Mood Disorder with Psychotic Features are partially or completely refractory to standard antipsychotic drug treatments, there are no adequate treatments for all symptoms of the disorders with one drug regimen. There are no effective treatments for negative symptoms which include amotivation, anhedonia, alogia, anergia, and affective impairment. There are no treatments for the progressive ventricular enlargement or cognitive declines that consistently accompany schizophrenia. Although many medical and psychopharmaceutical treatments have been patented for use with the above-identified neuropsychiatric diseases with varying degrees of success and side effects, there is no true understanding of the cause of the underlying biochemical nature of these disorders, nor any satisfactory long term treatment for it.

Schizophrenia has been treated with a variety of antipsychotic drugs such as phenothiazines, butyrphenones, xithioxanthenes, and newer atypical drugs, such as clozapine which are known to act as blockers of dopamine, serotonin, and cholinergic receptor sites. The modulation of a neurotransmitter receptor is the standard pharmacologic approach to treatment of schizophrenia. Bipolar disorder has been treated with anti-epileptic drugs and lithium which also block certain neurotransmitter receptors with limited efficacy.

Because of the inadequacy of present treatments for Schizophrenia, Bipolar Disorder I, Bipolar Disorder I with Psychotic Features, Bipolar Disorder II, Bipolar Disorder II with Psychotic Features, Psychotic Disorder Not Otherwise Specified, Schizophreniform Disorder, Schizoaffective Disorder, Unipolar Disorder, Unipolar Disorder with Psychotic Features, Substance Induced Psychotic Disorder, Schizotypal Personality Disorder and Mood Disorder with Psychotic Features, there exists a need for a more effective treatment. The present invention overcomes the previous limitations and shortcomings in the art by providing a novel way of treating the diseases by providing a method of inhibiting endogenous proteases and neuraminidases which convert N-CAM into cN-CAM and other breakdown fragments in the brain and CSF. An unexpected discovery of the present invention is that the breakdown product of N-CAM assayed in brain and CSF is found in synapses and can be reduced with protease inhibitors and that neuraminidase treatment of brain induces less resistance to protease inhibition. Therapy that reduces the N-CAM breakdown products is useful in treating the neuropsychiatric disorders named herein. Also provided are methods of monitoring the efficacy of treatment and screening for effective therapeutic compounds which can be used alone or in combination with standard antipsychotic therapy.

### SUMMARY OF THE INVENTION

The present invention provides a method of treating a neuropsychiatric disease in a human subject comprising administering a therapeutically effective amount of at least one compound selected from the group consisting of protease inhibitors and neuraminidase inhibitors, whereby administering the compound to the subject treats the neuropsychiatric disease.

Further, the present invention provides a method of reducing breakdown of N-CAM into a fragment in a brain of a human subject, comprising administering a therapeutically effective amount of a composition comprising at least one compound selected from the group consisting of protease inhibitors and neuraminidase inhibitors, whereby administering the composition to the subject reduces the breakdown of N-CAM into the fragment in the brain of the subject.

The present invention provides a method of treating a neuropsychiatric disease in a human subject comprising administering a therapeutically effective amount of a compound which reduces breakdown of endogenous protease inhibitors, whereby administering the compound to the subject treats the neuropsychiatric disease.

Further, the present invention provides a method of monitoring efficacy of treatment of a neuropsychiatric disease in a human subject, comprising detecting a reduction in concentration of a fragment of a neural cell adhesion molecule in cerebrospinal fluid of the subject, whereby the reduction in concentration of the fragment of the neural cell adhesion molecule in cerebrospinal fluid of the subject indicates efficacy of treatment of the neuropsychiatric disease.

Moreover, the present invention provides a method of screening for a compound effective in treating a neuropsychiatric disease associated with the presence of an abnormally elevated level of a fragment of a neural cell adhesion molecule in cerebrospinal fluid of a human subject, comprising the following steps: a) contacting a sample of human brain cortex with the compound and b) detecting a reduction of breakdown of the neural cell adhesion molecule into the fragment, whereby the reduction of breakdown of the neural cell adhesion molecule into the fragment indicates that the compound is effective in treating the neuropsychiatric disease.

Also provided by the present invention is a fragment of N-CAM, wherein the fragment is selected from the group consisting of dN-CAM, VASE N-CAM 155, VASE N-CAM 165 and VASE N-CAM 200.

The present invention also provides a method of treating a neuropsychiatric disease in a human subject comprising administering a therapeutically effective amount of a compound which reduces breakdown of endogenous protease inhibitors, whereby administering the compound to the subject treats the neuropsychiatric disease.

The present invention also provides a method of screening for a compound effective in reducing the breakdown of endogenous protease inhibitors in human brain cortex, comprising the following steps: a) contacting a sample of brain cortex with the compound; and b) detecting a rise in level or maintenance of a steady-state level of endogenous protease inhibitor, whereby the rise in the level or maintenance of a steady-state level of endogenous protease inhibitor indicates that the compound is effective in reducing the breakdown of endogenous protease inhibitors in the brain. A “steady-state level” means that the level of protease inhibitor does not fall from the initial baseline measurement.

The present invention provides a composition comprising a protease inhibitor and a neuraminidase inhibitor in a pharmaceutically acceptable carrier.

Various other objectives and advantages of the present invention will become apparent from the following detailed description.

### BRIEF DESCRIPTION OF THE DRAWINGS

FIG. 1—The effect of protease inhibitors on cortical synaptosome preparation from human brain. (A) The synatposomes were identified by synaptophysin immunoreactivity and also analyzed for N-CAM by SDS-PAGE and immunoblotting. The protease inhibitor was removed from the synaptosomal preparation prior to denaturing and running on a gel. The results show a decrease in N-CAM 180 and increase in cN-CAM. Lane numbers refer to the sucrose density gradient fractions. (B) Quantification of the immunodensity results show the protease inhibitors decrease the amount of cN-CAM relative to N-CAM 180 in each synaptosomal brain fraction.

FIG. 2—The effect of protease on proteolysis of N-CAM 180 in human cortex. (A). Cytosolic extracts of cortical samples were left at room temperature for 4 or 48 hr and treated with protease inhibitors (+) or no protease inhibitor (−). Membrane extracts of brain samples were similarly treated. The results show that membrane N-CAM 180 is predominantly converted to cytosolic N-CAM at 48 hrs. Without protease inhibitors the proteolysis is not halted and all N-CAM 180 isoforms are degraded by proteolysis. (B). The quantification shows that the cN-CAM and N-CAM 180 are unchanged in frozen samples; however, as the proteolysis reaction proceeds at 4 hr and 48 hr there is a progressive loss of N-CAM 180 and build-up of cytosolic N-CAM (cN-CAM). The maximally effective action of the protease inhibitor appears at the 4 hr time point in this experiment.

FIG. 3—High density particles from sucrose density ultracentrifugation of cytosolic brain were obtained at a sucrose density of 1.194 g/L (FIG. 3A) and showed N-CAM immunoreactivity. The step gradient also showed synaptophysin immunoreactivity that co-migrated with cN-CAM (FIG. 3B). Pretreatment of the brain cytosol with NP-40 prior to ultracentrifugation altered the migration of N-CAM in sucrose density gradient (FIG. 3C). Approximately 50% of cN-CAM stayed at the top of the gradient following NP-40 pretreatment and the remaining sedimented to the 1.237 g/L density. The NP-40 pretreatment disrupted the migration of synaptophysin in the sucrose gradient (FIG. 3D) so that synaptophysin was seen in low concentration in all sucrose fractions, due to membrane fragmentation.

FIG. 4—The control fraction from the brain sucrose density preparations (PBS treatment only) was visualized by fluorescent immunocytochemistry for N-CAM (FIG. 4A) and synaptophysin (FIG. 4B).

FIG. 5—Electron microscopy results show irregular clusters of densely packed aggregated spheres and membranous particles.

FIG. 6—The correlation of cN-CAM and changes in ventricular volume as measured by repeat MRI scans at a two-year interval. Patients with higher initial cN-CAM concentration showed higher ventricular enlargement. Ventricular enlargement is the most widely replicable indicator of brain differences between patients with schizophrenia and age-matched controls. Ventricular enlargement is believed to be an indicator of a progressive illness and a more severe illness often refractory to standard treatment.

FIG. 7—The effect of neuraminidase on conversion of N-CAM 180 to cN-CAM. (A). Neuraminidase enzyme treatment (+) of membrane and cytosolic human brain extracts is shown to be permissive for conversion of N-CAM 180 to a low molecular weight N-CAM isoform (dN-CAM). cN-CAM is partially glycosylated as shown in a small band migrating ahead of the majority of cN-CAM. cN-CAM neuraminidase treatment also produced a small amount of dN-CAM that is visible on the original film. N-CAM 75 kDa is also shown to be glycosylated and converted to dN-CAM. (B). The main effect of neuraminidase treatment is that the deglycosylation of N-CAM 180 permits proteolytic processing of N-CAM 180 to cN-CAM and dN-CAM isoforms.

FIG. 8—Accumulation of cN-CAM in brain samples as a function of time of incubation at room temperature. Data shown are amounts of N-CAM present as a percentage of the amount present at 0 hours. Circles indicate cN-CAM (105-115 kDa) and squares indicate N-CAM 180 kDa, the predominant membrane-associated form. (A) Accumulation of cN-CAM (circles) over the course of 48 hr of incubation, corresponding to a 25% decrease in concentrations of 180 kDa N-CAM in the control condition over the same time period. These samples contained low levels of a cocktail of protease inhibitors, in which the samples were customarily stored. (B and C) Both leupeptin and antipain completely inhibited the increase in cN-CAM (circles) otherwise seen over the course of 48 hr.

FIG. 9—Neuroserpin gene expression in postmortem brain tissue specimens from the prefrontal cortex in schizophrenia, expressed as a z-ratio. Z-ratio values are based on the distribution of z-score differences among a series of 1128 genes evaluated by microarray analysis. A z-ratio of 1 indicates that the difference is one standard deviation above the mean difference between patients with schizophrenia and controls. A z-ratio of negative 1 indicates a decrease of one standard deviation below the mean difference between schizophrenia and the matched control group. A z-ratio change of negative 2 represents approximately an 88% reduction in relative gene expression levels between groups. Each pool of samples contains a separate matched group of five patients with schizophrenia and five controls. A total of 20 patients and 20 controls is represented by the average z-ratio.

### DETAILED DESCRIPTION OF THE INVENTION

As used herein, “a” “an” or “the” may mean one or more. For example, “an” inhibitor may mean one inhibitor or more than one inhibitor. Moreover, “the” fragment may mean one fragment or more than one fragment.

The present invention provides a method of treating a neuropsychiatric disease in a human subject comprising administering a therapeutically effective amount of at least one compound selected from the group consisting of protease inhibitors and neuraminidase inhibitors, whereby administering the compound to the subject treats the neuropsychiatric disease. A “neuropsychiatric” disease is one which affects the neurologic system or the mind of a human subject. A neuropsychiatric disease may cause any of the following symptoms of psychosis: delusions, hallucinations, disorganized speech, grossly disorganized or catatonic behavior, negative symptoms. Other symptoms of neuropsychiatric disease are known to a person skilled in the art. Examples of a neuropsychiatric disease which are treatable with the present invention include but are not limited to Schizophrenia, Bipolar Disorder I, Bipolar Disorder I with Psychotic Features, Bipolar Disorder II, Bipolar Disorder II with Psychotic Features, Psychotic Disorder Not Otherwise Specified, Schizophreniform Disorder, Schizoaffective Disorder, Unipolar Disorder, Unipolar Disorder with Psychotic Features, Substance Induced Psychotic Disorder, Schizotypal Personality Disorder and Mood Disorder with Psychotic Features. These diseases are usually “chronic.” A chronic disease is one which is present in a subject and manifests its signs and symptoms for at least 3 to 6 months.

For example, schizophrenia is a life-long illness associated with increased ventricle size of the brain, thinning of the cortical grey matter and cognitive decline. Symptoms are grouped into three categories: positive or expressive symptomatology, negative or deficit symptomatology and disorganized symptomatology further defined in DSM-IV. The disease may be diagnosed after a period of time known as the prodrome, which may last from a few months up to two years. During the prodrome of schizophrenia, the subject manifests early symptoms suggesting the presence of a neuropsychiatric disease. However, a conclusive diagnosis of schizophrenia is not usually made for several months. During the active phase, a subject will manifest positive, negative or disorganized symptomatology for a significant portion of time during a 1 month period. The present invention is directed to the treatment of subjects with chronic schizophrenia who have manifested some symptoms of the disease for approximately six months.

After approximately six months following the diagnosis of schizophrenia and after an initial psychotic episode, abnormally elevated levels (concentrations) of a fragment of a neural cell adhesion molecule are found in the CSF of the subject. A “neural cell adhesion molecule” is one found in a synaptosome in the synaptic region of a neuron in the brain and functions to facilitate neurite growth, neuronal migration, signaling and transmission of impulses across the synaptic junction between neurons in the brain.

Thus, the inherent disease process of schizophrenia itself appears to be responsible for increases in cN-CAM in CSF since there are no abnormalities detected in untreated first episode patients or normal monozygotic twins, but changes are later detected in brain tissue and CSF of chronic schizophrenic subjects. It is conceivable that minor abnormalities in the brain present at the onset of the disease are not detectable in CSF, but nevertheless would be present in brain if there were a window to probe human brain at the time of onset, which is not likely with present technology. As schizophrenia progresses, the CSF levels of cN-CAM increase. Furthermore, a progression of symptoms is associated with other N-CAM fragments, and ventricular enlargement is associated with cN-CAM. Moreover, males appear more susceptible to increasing levels of cN-CAM in CSF as compared to females, which is in line with the severity of the disease and illness progression of male patients observed through the decades of clinical observations.

The present invention treats neuropsychiatric diseases characterized by the presence of an elevated level of proteolytic breadkdown products of an isoform of N-CAM. The breakdown products are fragments of the isoform of N-CAM and are present at abnormally elevated levels in bipolar disorder which is characterized clinically by abnormal affect related to excessive swings in mood between depression, euthymia and mania. Fragments of N-CAM elevated in bipolar disorder are VASE N-CAM and SEC N-CAM in the brain.⁷ Moreover, cN-CAM is elevated in bipolar disorder I.

Thus, the present invention provides a method of reducing breakdown of N-CAM into a fragment in a brain of a human subject, comprising administering a therapeutically effective amount of a composition comprising at least one compound selected from the group consisting of protease inhibitors and neuraminidase inhibitors, whereby administering the composition to the subject reduces the breakdown of N-CAM into the fragment in the brain of the subject. Reducing the breakdown of N-CAM into fragments in the brain or CSF thereby treats the neuropsychiatric disease.

The present invention is based on the unexpected and surprising discovery that reducing the breakdown of a neural cell adhesion molecule in a brain of a subject affected with a neuropsychiatric disease improves the cognitive and psychiatric functions of the subject. The present invention provides a method of reducing the proteolytic breakdown of N-CAM, a neural cell adhesion molecule, into fragments associated with the presence of a neuropsychiatric disease.

In normal brain metabolism, endogenous proteases and neuraminidases degrade an isoform of N-CAM and release fragments of the isoform into CSF. Isoforms of N-CAM are N-CAM 180, N-CAM 140, N-CAM 120, SEC N-CAM 115 and VASE N-CAM 200. Fragments produced by the proteolytic breakdown of the isoforms of N-CAM are cN-CAM, dN-CAM, SEC N-CAM 108,VASE N-CAM 165 and VASE N-CAM 155. Although normal subjects have fragments of N-CAM in CSF, subjects with chronic schizophrenia have levels of fragments which are approximately 135% to 200% of normal levels.

Further, the present invention provides methods to treat neuropsychiatric diseases characterized by symptoms described above for “neuropsychiatric” disorders similar to schizophrenia but include Psychotic Disorder, Schizophreniform Disorder, Schizoaffective Disorder, Mood Disorder with Psychotic Features, Substance Induced Psychotic Disorder and Schizotypal Personality Disorder. In these disorders there is an abnormally elevated level of fragments of an isoform of N-CAM in the CSF of affected subjects. “Elevated” means characterized by an increase in the amount present compared to an age-matched control or a pre-disease state in the subject.

Moreover, the present invention provides a method of treating a neuropsychiatric disease characterized as a psychosis. The psychosis is usually chronic and is characterized by the presence of one or more of the following: delusions, hallucinations, disorganized speech, and grossly disorganized or catatonic behavior. However, an acute drug-induced exacerbation of a chronic psychosis caused by the subject's long-term drug abuse may also be treated by the method of the present invention. In these disorders there is an abnormally elevated level of fragments of an isoform of N-CAM in the CSF of affected subjects.

The present invention is directed at reducing the proteolytic breakdown in the synaptosome of an isoform of a neural cell adhesion molecule known as N-CAM by inhibiting endogenous proteases and neuraminidases. It is the excessive breakdown of N-CAM into fragments that is associated with the presence of the neuropsychiatric diseases. Examples of endogenous proteases include but are not limited to serine proteases, aspartyl proteases, tissue plasminogen activator, metalloproteinases, aminopeptidases and cysteine proteases. Moreover, examples of endogenous neuraminidases include but are not limited to neuraminidase 1, neuraminidase 2 and neuraminidase 3.

The present invention discloses a method of reducing the proteolytic breakdown of N-CAM, comprising either administering a compound which inhibits the enzymes responsible for degrading N-CAM or administering a compound which either elevates the level and activity of endogenous protease inhibitors and neuraminidase inhibitors or which reduces the breakdown of the endogenous protease inhibitors and neuraminidase inhibitors. For example, the main protease inhibitor target in the brain is tissue type plasminogen activator (t-PA), a serine protease. Because t-PA degrades N-CAM, t-PA is a prime target for treatment with protease inhibitors and compounds of the invention.

Moreover, t-PA activity in the central nervous system is regulated by the endogenous protease inhibitors known as serpins. Examples of serpins are plasminogen activator inhibitor (PAI-1), protease nexin-1 (PN-1), and neuroserpin (NSP)¹. It is contemplated in the present invention that a serpin will be administered to a subject with neuropsychiatric disease to reduce the proteolytic breakdown of N-CAM by endogenous proteases. Moreover, in another embodiment of the present invention, a compound which increases the level of a serpin or reduces breakdown of a serpin, thereby inhibiting breakdown of N-CAM, can be administered to a subject with a neuropsychiatric disease. Furthermore, nucleic acids encoding serpins can be administered to a subject with neuropsychiatric disease, as can nucleic acids that encode compounds that reduce the breakdown of serpins or nucleic acids that themselves interfere with the breakdown of serpins (e.g., an antisense nucleic acid directed to block a nucleic acid which encodes a protease).

There are several classes of protease inhibitors. See Table 1. Examples of aspartic protease inhibitors (A) include Nelfinavir, Saquinavir, Indinavir, Amprenavir, Ritonavir, Pepstatin, AG1776, ABT-387 and β-secretase inhibitors.

Serine protease inhibitors (B) examples include Aprotinin, AEBSF, Leupeptin (Acetyl-leucyl-leucyl-arginal), Elastatinal (Leu-(Cap)-Gln-Ala-al, N-[(S)-1carboxy-isopentyl)-carbamoyl-alpha-(2-iminohexahydro-4(S)-pyrimidyl]-L-glycyl-L-glutaminyl-L-alaninal), Serpins, Antipain ([(S)-1-Carboxy-2-Phenyl]-carbamoyl-Arg-Val-arginal), APMSF ((4-Amidino-Phenyl)-Methane-Sulfonyl Fluoride) and PMSF.

Cysteine protease inhibitors (C) examples include AG7088, Leupeptin and E-64 (L-trans-epoxysuccinyl-leucylamide-(4-guanido)-butane or N-[N-(L-trans-carboxyoxiran-2-carbonyl)-L-leucyl]-agmatine).

Aminopeptidase inhibitors (D) examples include Bestatin ([(2S, 2R)-3-Amino-2-hydroxy-4-Phenylbutanoyl]-L-Leucine) and Amastatin ([(2S, 2R)]-3-Amino-2-hydroxy-5-methylhexanoyl]-Val-Val-Asp-OH).

Moreover, Table 1 shows examples of neuraminidase inhibitors (E) including GS 4071, GS 4104, oseltamivir, zanamivir and RWJ-270201. Table 1 shows which compounds are currently approved by the FDA and which are not yet approved. Moreover, the underlined compounds are components of the protease inhibitor cocktail.

The present invention provides a “protease inhibitor cocktail” comprising the following compounds:

1) Product Name: Antipain

Synonyms: [(S)-1-Carboxy-2-phenylethyl]carbamoyl-L-arginyl-L-valyl-argininal

Literature References: Protease inhibitor: H. Suda, et al., J. Antibiotics 25, 263 (1972)

OR

Product Name: Antipain Hydrochloride

Synonyms: N-(-Nalpha-Carbonyl-Arg-Val-Arg-al)-Phe

Non-selective serine and cysteine protease inhibitor

Literature References: Suda, H., et al., J. Antibiot., 25, 263 (1972);

2) Product Name: Pepstatin A

Synonyms: Isovaleryl-L-valyl-L-valyl-[(3S,4S)-4-aminio-3-hydroxy-6-methylheptanoyl]-L-alanyl[(3S,4S)-4-amino-3-hydroxy-6-methylheptanoic acid]

OR

CAS: 26305-03-3

Literature References: Strong inhibitor of acid proteases—pepsin, cathepsin D, renin: H. Umezawa, Methods Enzymol. 45, 689 (1976); H. Umezawa, Acta Biol. Med. Germ. 36, 1899 (1977); J. Tang, Nature 266, 119 (1977);

3) Product Name: Aprotinin from bovine lung

Synonyms: Trypsin Inhibitor (basic, pancreatic);

4) Product Name: Leupeptin Hydrochloride

Synonyms: Acetyl-Leu-Leu-Arg-al

Literature References: Aoyagi, T., J. Antibiot., 22, 283 (1969); Saino, T., et al., Chem.

Pharm. Bull., 30, 2319 (1982); and

5) Product Name: Phenylmethylsulfonyl fluoride

Comments: Inhibitor of serine proteases such as trypsin and chyrnotrypsin, (Moss, D. E. and Fahmey, D. E., Biochemical Pharmacology, 27, 2693, 1978) and of mammalian acetylcholinesterase, (Turini, P., et al., J. Pharmacol. Exp. Ther., 167, 98, 1969).

The present patent provides numerous examples of protease inhibitors and neuraminidase inhibitors for use in the compositions and methods of the invention. It is understood, however, that any protease inhibitor or neuraminidase inhibitor now known or later developed can be routinely screened for efficacy in the present methods or can be included in the present compositions.

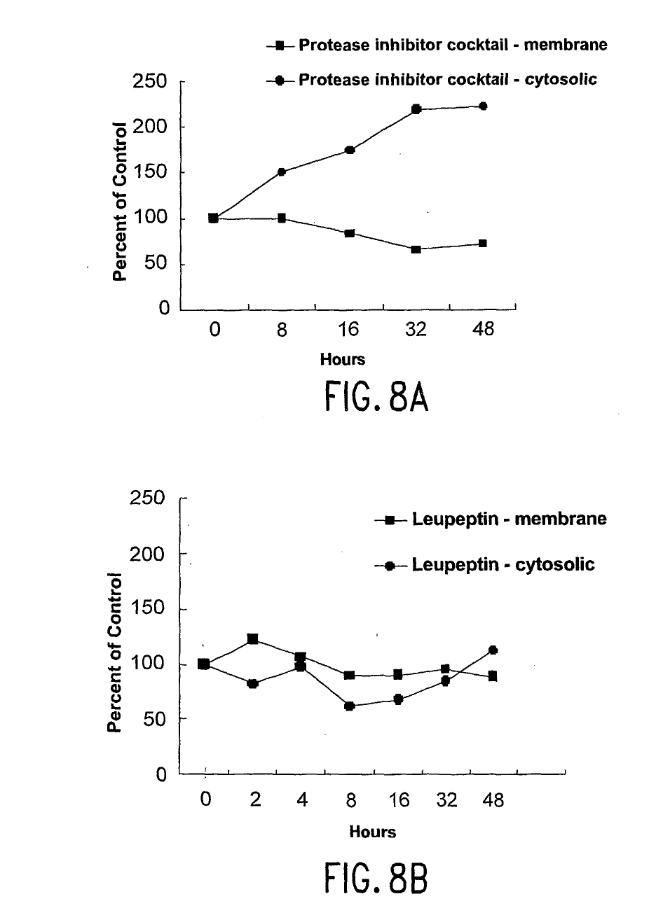

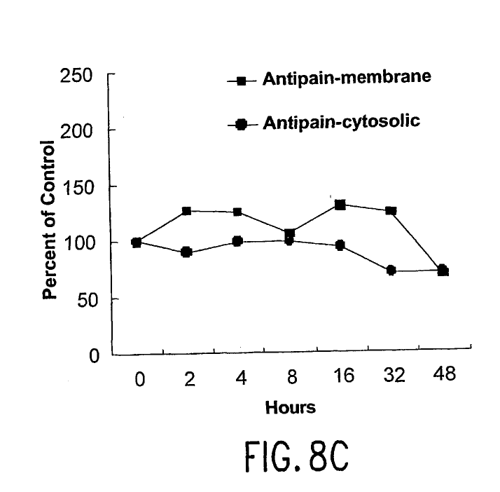

While numerous protease inhibitors are effective in the present methods, antipain and leupeptin are the two protease inhibitors most effective in preventing the breakdown of N-CAM 180 and the accumulation of cN-CAM (FIG. 8). These compounds inhibit both serine and cysteine proteases. When assayed in human brain, antipain and leupeptin are the most potent compounds compared to the protease inhibitor cocktail. See Example 9.

In the present invention, the protease inhibitor or neuraminidase inhibitor can be administered to a subject in a pharmaceutically acceptable carrier. By “pharmaceutically acceptable” is meant a carrier that is not biologically or otherwise undesirable, i.e., the carrier may be administered to a subject, along with the protease inhibitor or neuraminidase inhibitor, without causing any undesirable biological effects or interacting in a deleterious manner with any of the other components of the pharmaceutical composition in which it is contained. The carrier would naturally be selected to minimize any breakdown of the active ingredient and to minimize any adverse side effects in the subject, as would be well known to one of skill in the art.

The protease inhibitor or neuraminidase inhibitor may be administered orally, parenterally (e.g., intravenously), by intramuscular injection, by intraperitoneal injection, transdermally, extracorporeally, intrarectally, intrathecally, by inhalation into the respiratory tract, topically or the like, although oral administration is typically preferred. Dosage ranges and preferred dosages and routes of administration for FDA approved protease inhibitors and neuraminidase inhibitors are shown in Table 2. The exact amount of the protease inhibitor or neuraminidase inhibitor required will vary from subject to subject, depending on the age, weight and general condition of the subject, the severity of the disease or condition being treated, the particular protease inhibitor or neuraminidase inhibitor used, its mode of administration and the like. Thus, it is not possible to specify an exact amount of protease inhibitor or neuraminidase inhibitor to administer. However, an appropriate amount can be determined by one of ordinary skill in the art using only routine experimentation given the teachings herein². Examples of dosage ranges and preferred dosages and routes of administration for FDA approved protease inhibitors and neuraminidase inhibitors are shown in Table 2.

Parenteral administration of the protease inhibitor or neuraminidase inhibitor of the present invention, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

Suitable carriers for use in the present invention include, but are not limited to, pyrogen-free saline. For parenteral administration, a sterile solution or suspension is prepared in saline that may contain additives, such as ethyl oleate or isopropyl myristate, and can be injected, for example, into subcutaneous or intramuscular tissues.

Suitable carriers for oral administration of a protease inhibitor or neuraminidase inhibitor include one or more substances which may also act as flavoring agents, lubricants, suspending agents, or as protectants. Suitable solid carriers include calcium phosphate, calcium carbonate, magnesium stearate, sugars, starch, gelatin, cellulose, carboxypolymethylene, or cyclodextrans. Suitable liquid carriers may be water, pharmaceutically accepted oils, or a mixture of both. The liquid can also contain other suitable pharmaceutical additions such as buffers, preservatives, flavoring agents, viscosity or osmo-regulators, stabilizers or suspending agents. Examples of suitable liquid carriers include water with or without various additives, including carboxypolymethylene as a pH-regulated gel.

Alternatively, the protease inhibitor or neuraminidase inhibitor may be microencapsulated with either a natural or a synthetic polymer into microparticles 4-8 μm in diameter, which target intestinal lymphoid tissues and produce a sustained release for up to four weeks.

In Table 3, dosage ranges and preferred dosages and routes of administration for investigational new drugs (IND) contemplated for use in the compositions and methods of the present invention are shown. Furthermore, Tables 4 and 5 show compounds which are in pre-clinical testing and the dosages and routes of administration for those drugs.

The present invention comprises administering a protease inhibitor or a neuraminidase inhibitor to a subject diagnosed with a neuropsychiatric disease. Moreover, the present invention includes administering a protease inhibitor and a neuraminidase inhibitor in combination to a subject diagnosed with a neuropsychiatric disease. Further, either a protease inhibitor or a neuraminidase inhibitor or a combination of both may be administered with a neuroleptic compound to a subject with neuropsychiatric disease. A “neuroleptic” compound is an antipsychotic medication and includes typical neuroleptics such as dopamine antagonists, for example Haldol® and other drugs which block dopamine receptors in the brain. An example of an atypical neuroleptic compound is clozapine which blocks other receptors as well as some dopamine receptors in the brain. Neuroleptics have a beneficial effect on the positive and negative symptomatology of chronic schizophrenia but do not improve the cognitive function of affected subjects. A benefit of the present invention is the improvement in cognitive function of an affected subject.

The present invention provides a method of monitoring the efficacy of treatment of a neuropsychiatric disease in a human subject comprising detecting a reduction in concentration (level) of a fragment of a neural cell adhesion molecule in CSF of the subject, whereby the reduction in concentration of the fragment of the neural cell adhesion molecule in cerebrospinal fluid of the subject indicates efficacy of treatment of the neuropsychiatric disease. After the initiation of therapy with the method of the present invention, a person of skill in the art will follow the progress of the treatment by noting a decrease in the positive and negative symptomatology in a subject with a neuropsychiatric disease. Moreover, the person of skill in the art will detect improvement in the cognitive function of the subject. For example, a person with chronic schizophrenia may be noted to have fewer delusions, fewer hallucinations, fewer negative symptoms, less disorganized speech, and less grossly disorganized or catatonic behavior, as measured by psychometric tests known and used routinely by persons skilled in the art.

Further, the present invention provides a laboratory method of monitoring the efficacy of treatment of the present invention. At the time of diagnosis of the neuropsychiatric disease, a baseline measurement of the level of fragments in the CSF of the subject is made. The fragments to be measured comprise cN-CAM, dN-CAM, SEC N-CAM 108, VASE N-CAM 165 and VASE N-CAM 155. After three months of therapy with the method of treatment of the present invention, a repeat lumbar puncture is performed to obtain the subject's CSF to measure the level of fragments in it. Clinical improvement in the subject correlates with a significant reduction in the level of fragments in the CSF. A subject with a baseline pretreatment level of fragments 135% of normal would be expected to show a reduction of fragments to no more than 117% of normal. Moreover, a subject with more severe clinical disease, correlated with a baseline pretreatment level of fragments of 200% of normal, would be expected to show a reduction in fragment level in the CSF to approximately 140% to 160% of nonnal. A method of monitoring the efficacy of treatment is disclosed in Example 7. For patients with intermediate levels of elevation, the reduction will range from about 40-60%.

The present invention also provides a method of screening for a compound effective in treating a neuropsychiatric disease associated with the presence of an elevated level of a fragment of a neural cell adhesion molecule in brain or CSF of a human subject, comprising the following steps: a) contacting a sample of brain cortex with the compound, and b) detecting a reduction of breakdown of the neural cell adhesion molecule into the fragment, whereby the reduction of breakdown of the neural cell adhesion molecule into the fragment indicates that the compound is effective in treating the neuropsychiatric disease. The brain tissue may be pulverized, sonicated, homogenized or dissolved in solution prior to contacting with the compound. Moreover, any physical process that allows brain cortex to come into contact with a compound for in vitro testing of N-CAM breakdown into fragments is contemplated by the present invention. See Example 1.

Moreover, cN-CAM is present in sub-human primate (for example, squirrel monkey) brain and cerebrospinal fluid. Thus, primates can be used as a model for studies on the effects of protease inhibitors on cN-CAM and N-CAM 180 kDa levels. See Example 8.

The present invention provides a method of screening for compounds which are effective in treating neuropsychiatric diseases characterized by the abnormal elevation of fragments of N-CAM in the brain or CSF of a human subject. The present screening methods can be used to screen for the neuropsychiatric diseases described herein. See Example 6.

Examples of chronic neuropsychiatric diseases which are characterized by abnormally high levels of fragments of an isoform of the neural cell adhesion molecule N-CAM are Schizophrenia, Bipolar Disorder I, Bipolar Disorder I with Psychotic Features, Bipolar Disorder II, Bipolar Disorder II with Psychotic Features, Psychotic Disorder Not Otherwise Specified, Schizophreniform Disorder, Schizoaffective Disorder, Unipolar Disorder, Unipolar Disorder with Psychotic Features, Substance Induced Psychotic Disorder, Schizotypal Personality Disorder and Mood Disorder with Psychotic Features. Isoforms of N-CAM comprise N-CAM 180, N-CAM 140, N-CAM 120, SEC N-CAM 115 and VASE N-CAM 200. A subject with a history of a chronic psychosis caused by long-term drug abuse (e.g., methamphetamine) may be found to have an abnormally elevated level of fragments in the CSF during an acute psychotic reaction from a drug overdose. Moreover, an acute exacerbation of a dementia associated with delusions and hallucinations is a disease which can be diagnosed by finding abnormally elevated levels of fragments of N-CAM. The fragments include cN-CAM, dN-CAM, SEC N-CAM 108, VASE N-CAM 165 and VASE N-CAM 155. A person skilled in the art has the knowledge to perform a lumbar puncture to obtain CSF for laboratory analysis. The procedure is performed routinely by medical practitioners to diagnose diseases of the brain and spinal cord.

The present invention provides a fragment of a neural cell adhesion molecule selected from the group consisting of dN-CAM, VASE N-CAM 155, VASE N-CAM 165 and VASE N-CAM 200.

The present invention also provides a method of treating a neuropsychiatric disease in a human subject comprising administering a therapeutically effective amount of a compound which reduces breakdown of an endogenous protease inhibitor in brain, whereby administering the compound to the subject treats the neuropsychiatric disease. A compound that reduces the breakdown of endogenous protease inhibitors will cause the level of endogenous protease inhibitors in brain to rise or remain steady. Thus, the breakdown of N-CAM into fragments associated with the neuropsychiatric diseases described herein will be reduced. Therefore, the neuropsychiatric disease is treated.

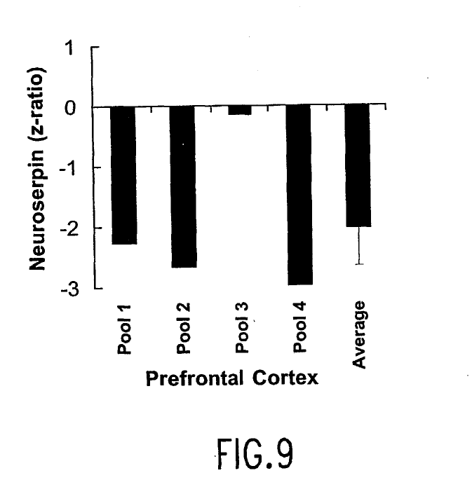

Messenger RNA (mRNA) for the endogenous serine protease inhibitor neuroserpin (gene product P112, Genbank Accession #Z81326) is decreased in the prefrontal cortex of patients with schizophrenia, as compared to controls FIG. 9). It has also been found that expression of neuroserpin mRNA is decreased by approximately 88% in patients with schizophrenia, as measured in four separate cohorts of patients. See Example 10. Thus, the methods of the present invention which counteract the defects in protease inhibition will decrease the buildup in the brain of cN-CAM and other breakdown products of N-CAM that are associated with schizophrenia.

Also provided is a method of screening for a compound effective in reducing the breakdown of endogenous protease inhibitors in human brain cortex, comprising the following steps: a) contacting a sample of brain cortex with the compound, and b) detecting a rise in level or maintenance of a steady-state level of an endogenous protease inhibitor, whereby the rise in the level or maintenance of the steady-state level of the endogenous protease inhibitor indicates that the compound is effective in reducing the breakdown of endogenous protease inhibitors in the brain. After baseline levels of endogenous protease inhibitors are determined in samples of brain cortex, some brain samples are contacted with a test compound and other brain samples, are controls. Finding elevated or maintained levels of endogenous protease inhibitors in the samples of brain contacted with the compound compared to controls indicates that the compound reduces the breakdown of endogenous protease inhibitors and is effective in treating neuropsychiatric diseases.

Moreover, the present invention provides a composition comprising a protease inhibitor and a neuraminidase inhibitor in a pharmaceutically acceptable carrier. The composition can include one or more protease inhibitors selected from the group of aspartic protease inhibitors, serine protease inhibitors, cysteine protease inhibitors and aminopeptidase inhibitors. Examples of the protease inhibitors include but are not limited to Nelfinavir, Saquinavir, Indinavir, Amprenavir, Ritonavir, Aprotinin, Pepstatin, AG1776, ABT-387, Beta-secretase inhibitors, AEBSF, Leupeptin, Elastatinal, Serpins, Antipain, APMSF, PMSF, AG7088, E-64, Betastatin and Amastatin. In addition to protease inhibitors, the composition can include one or more neuraminidase inhibitors. Examples of neuraininidase inhibitors include but are not limited to Zanamivir, Oseltamivir, RWJ-270201, GS 4071 and GS 4104. Suggested dosages and routes of administration of the protease inhibitors and neuraminidase inhibitors are shown in Tables 2, 3 and 4.

### EXAMPLE 1

cN-CAM Localizes in Human Brain Synaptosomes.

Procedure for localization of N-CAM in fresh human occipital cortex by preparation of a synaptosome suspension is described:

A section from the right human occipital cortex (49.4 g) was obtained 12 hours post-mortem and stored overnight at 4° C., diluted 20% wt/vol in solution A (0.32 M sucrose solution with 1 mM of NaHCO3, 1 mM MgCl2, and 0.05 mM of CaCl2 with protease inhibitors) and homogenized by 12 strokes with a glass-Teflon homogenizer. The homogenate was filtered with a nylon mesh screen sieve and centrifuged at a low speed 1475×g. The supernatant was saved and the resultant pellet (P1) was resuspended with solution A and centrifuged at 755×g. This supernatant was combined with the first supernatant and the combined supernatants (S 1A) were centrifuged at 17,500×g. The resultant pellet (P2) containing synaptosomes and mitochondria was resuspended in solution B (solution B is 0.32M sucrose solution with 1 mM NaHCO3) and loaded into a sucrose density gradient of 0.85, 1.0, 1.2M and centrifuged for 2 hours at 100,000×g. The band between 1.0 and 1.2M sucrose which contains synaptosomes (SX) was saved as well as the other sucrose fractions S3 (top), S4 (0.85M), S5 (1.0M), and S6 (1.2M). The synaptosome band (SX) was combined with 4 volumes of solution B and centrifuged at 48,200×g for 20 min. The pellet (P3) was resuspended with solution C (0.16M sucrose with 6 mM Tris-HCl containing 0.5% Triton X-100) and centrifuged at 48,200×g. The supernatant was saved (S7) and the pellet (P3) was resuspended in solution B (S8) and loaded on another sucrose gradient density with density steps of 1.0 (S10), 1.5 (S11) and 2.0M (S 12), and centrifuged for 2 hours at 275,000×g. The band between 1.5 and 2.0M contains the post synaptic densities, and the floating band at the top contains the synaptosomal membranes (S9). Rotors are Sorvall SS-34 (17,500 g) and Beckman T40I rotors(100,000×g and 275,000×g).

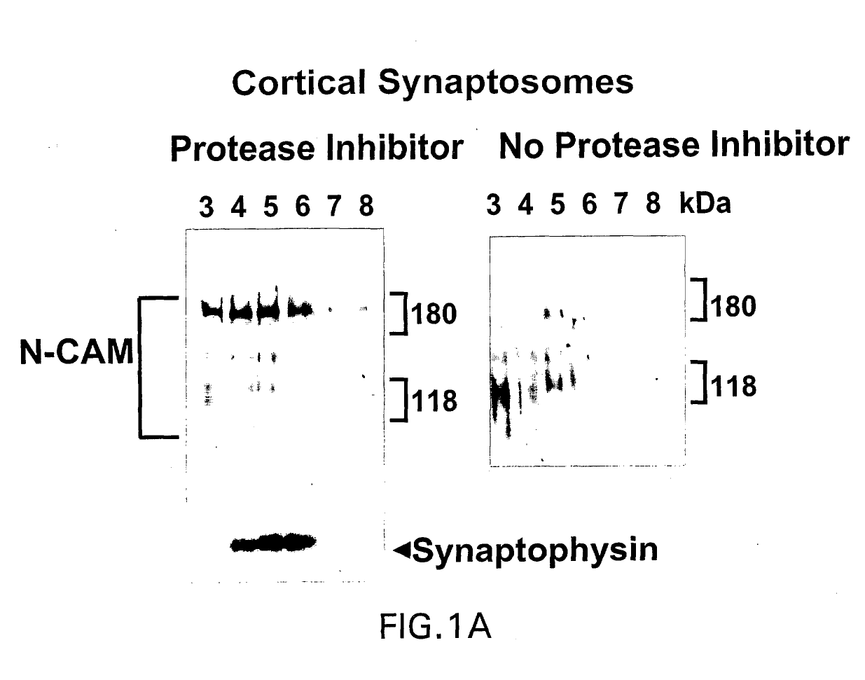

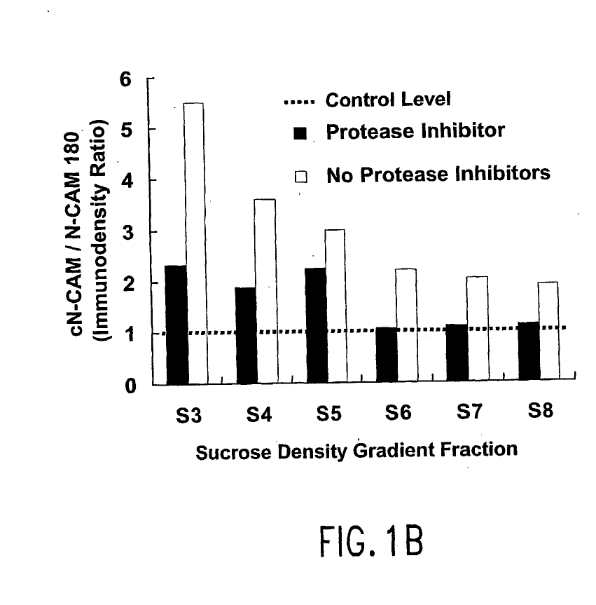

N-CAM 180 is found in both non-synaptosomal and synaptosomal fractions (lanes 3 versus 4, 5, 6 shown in FIGS. 1A and 1B). The synaptosome fraction is further delineated by synaptophysin reactivity in lanes 4, 5, 6. However, the breakdown fragment of N-CAM is also found in the synaptosome in both unconcentrated and concentrated samples, while the preponderance of N-CAM 180 disappears. The disappearance of N-CAM 180 can be quantified from the film images by calculating a ratio between the N-CAM 180 and N-CAM 105-115. Thus, enzyme breakdown is apparent in the synaptosomes as there is a decrease in the N-CAM 180/N-CAM 105-115 ratio following concentration in an Amicon filter (MW cut-off 3,000) and removal of the protease inhibitors.

### EXAMPLE 2

cN-CAM Occurs in Brain Via Proteolytic Mechanism Converting N-CAM 180 to cN-CAM as Shown Above in the Synaptosome.

Add liquid N₂ and grind up entire occipital cortex sample. Split into 6 equal samples while at the frozen state.

Prepare a soluble extract (described in Example 5) using regular PBS extraction buffer with protease inhibitors (condition A for 3 samples from each time-point). Use the other 3 samples for each time point and prepare a soluble extract without protease inhibitors (condition B). The 12 samples can be labeled as in Table 6.

Prepare a crude extract (described in Example 5) for 3 samples with protease inhibitors (one for each time point) and a crude extract for 3 samples without protease inhibitors (one for each time point).

Leave two samples out each at RT for 0 hrs, 4-8 hrs, and 48 hrs. Make the homogenates 20% wt/vol. with solutions from Table 6.

Save the soluble extracts separately (SA1, SB1, etc.). Resuspend the pellets as 20% solutions (use the same volumes of buffer as in soluble step) in PBS with and without protease inhibitors and keep them separate (CA1, CB1, CA2, CB2, CA3, CB3). Perform another centrifugation. Remove the supernatant and discard. To the remaining pellet perform the crude extraction (described in Example 5) with and without protease inhibitor. Recentrifuge after the 30 min extraction with stirring on ice. This experiment will yield a total of 12 samples that can fit on one gel. Load equal amounts of protein in each lane (40-60 micrograms). Use 20, 40, and 80 micrograms of a reference brain soluble and crude as the human standard. Split the immunoblot into a lower and upper half, e.g., between the pink and turquoise, since synaptophysin runs below the pink and soluble N-CAM (cN-CAM) around the turquoise. Use the Sigma high molecular weight standards (3312) for calibration. Incubate the lower half of the membrane with synaptophysin antibody, and the upper half with N-CAM 3732 antibody. Use standard immunoblot procedures. Take pictures with Kodak BioMax film.

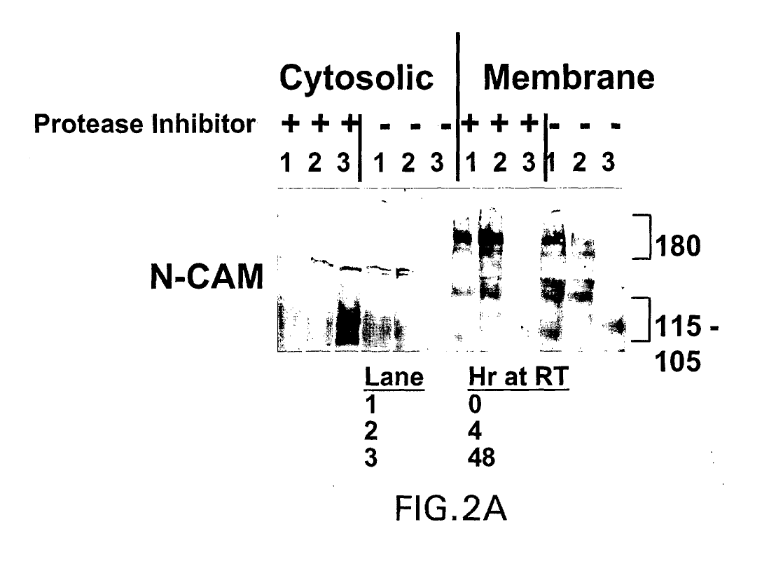

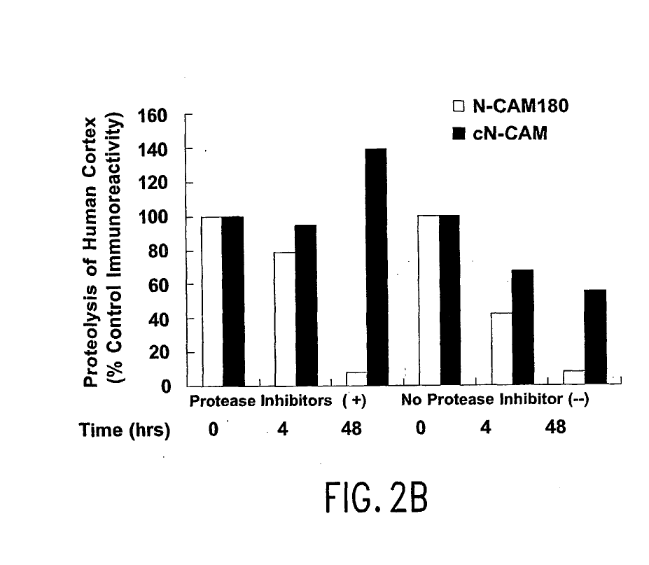

The proteolysis of both membrane and cytosolic N-CAM (cN-CAM) isoforms was monitored by Western immunoblotting. The results (FIGS. 2A and 2B) show that membrane N-CAM 180 isoform disappears upon incubation of tissue from 4 hrs -48 hrs, however N-CAM 105-115 kDa is preserved with protease inhibitors. Without protease inhibitors, there is no preservation of the N-CAM 105-115 kDa, in spite of breakdown of parent molecule N-CAM 180.

### EXAMPLE 3

cN-CAM is Colocalized to Particles in the Brain That Correspond to Synapses Without a Particular Limitation to a Type of Synapse.

Schizophrenia is associated with a high level of “immature” synapses defined as an elevated ratio of cN-CAM /synaptophysin levels³.

Ultracentrifugation of Brain

Cytosolic sample of brain homogenate (described below in Example 5) was ultracentrifuged for 120 min at 38,000 rpm in a Beckman SW40Ti rotor at 4° C. through a 0.5M, 1.0M and 1.2M sucrose density step gradient and analyzed for N-CAM and synaptophysin by Western immunoblot. A pellet was also obtained and analysed for N-CAM immunoreactivity by immunoblot. The pellet in 1.2M sucrose was resuspended and centrifuged for 60 min at 38,000 rpm at 4° C. in the Beckman SW40Ti rotor. The overlay of PBS was removed except approximately 0.5 ml of PBS and the resulting pellet was resuspended by pipetting up and down. A 100 microliter aliquot was treated with 1.9 ml of NP-40 detergent (5% solution in PBS, pH 7.4 with protease inhibitors) or control treatment consisted of 1.9 ml of PBS (phosphate buffered saline) with protease inhibitors. The tubes were then incubated at 4° C. on a rocker for 30 min. Each treated aliquot was overlaid on a step sucrose density gradient (4M, 3M, 2M, 1.5M, 1M sucrose) with protease inhibitors in PBS. The tubes were topped off with 0.3M sucrose and centrifuged at 30,000 rpm at 4° C. for 60 min. One ml fractions from each tube were removed and the absorbance at 280 nm measured against a 1M sucrose blank. Each sucrose fraction was run on a gel (70 μl sample and 30 μl 2× denaturing solution) and analyzed for N-CAM and synaptophysin.

The high density particles identified by ultracentrifugation of cytosolic brain were inununostained in chamber slides with antibodies to N-CAM and synaptophysin. All steps were performed at room temperature in an eight well chamber slide precoated with 0.1% BSA for 15 min, washed, and allowed to air dry in a dust free environment. An aliquot of the N-CAM enriched sucrose gradient sample was diluted 1: 10 in PBS and 2 μl was spotted into the center of each chamber. The slide was fixed with 20 μl of 5% paraformaldedyde in PBS pH 7.4 for 10 min and washed with 200 μl of PBS three times. N-CAM primary antibody (1:1000) and synaptophysin (1:1000) were added for 45 min at room temperature followed by three PBS washes and a final incubation with 100 μl of secondary antibody (1:250 anti-rabbit IgG FITC and anti-mouse rhodamine, Boehringer Mannheim Corp.) for 40 min followed by three PBS washes. The chambers were removed and an anti-fade solution (Molecular Probes, OR) was added prior to coverslip. The immunostained particles were imaged by confocal laser scanning microscopy (Zeiss Model 410, Germany).

Electron Microscopy

A suspension of particles from sucrose density gradient ultracentrifugation was fixed with 2% glutaraldehyde in PBS on ice for 20 min. The particles were pelleted at 10,000 g for 1 hour and the pellet was resuspended in 25 μl PBS and then mixed with 25 μl of 3% agarose at 60° C. A 1 μl droplet of agarose-particle mixture was solidified on parafilm, and cut into 1-2 mm cubes. The cubes were rinsed for 15 minutes three times in PBS and then fixed with 1% osmium tetroxide in PBS for 1 hour, and rinsed 3×10 min in PBS. The cubes were en block stained with 2% uranyl acetate for 1 hour, dehydrated through a series of 30%, 50%, 70%, 80%, 95%, and 100% ethanol solutions for 5 min each. The dehydrated cubes were infiltrated overnight at 55° C. with unaccelerated Durcupan ACM resin (Electron Microscopy Sciences, Fort Washington, Pa.), embedded into a flat mold with accelerated Durcupan resin and polymerized at 55° C. for 72 hours. Ultrathin sections of 90 nm from two different cubes were prepared on an Ultracut E ultramicrotome. The sections were stained with uranyl acetate and lead citrate, and viewed with Zeiss 600 EM10A transmission electron microscope at 60 kV.

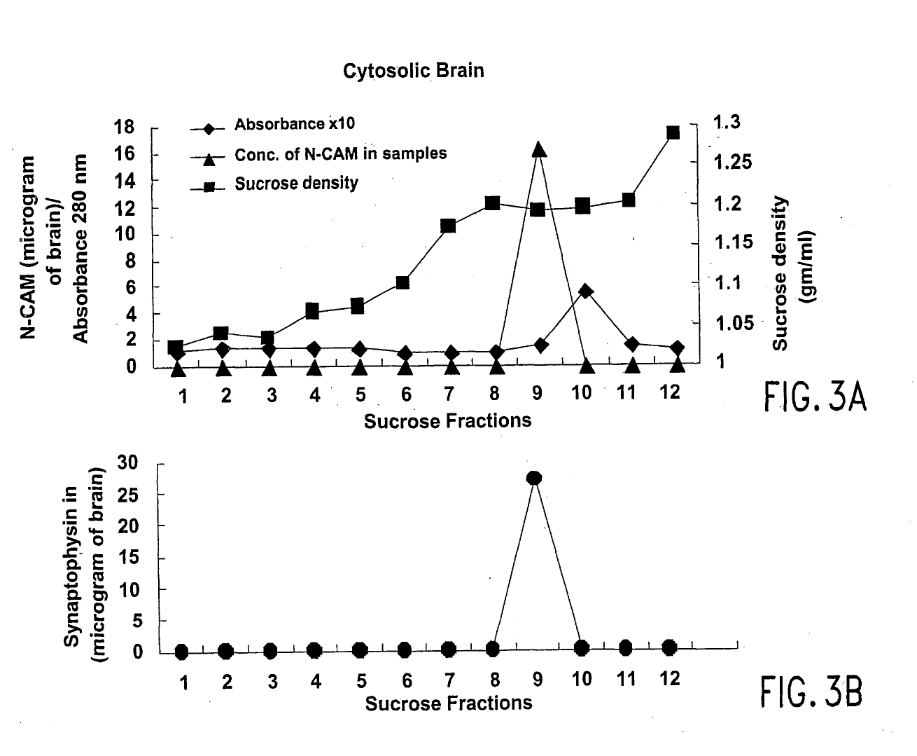

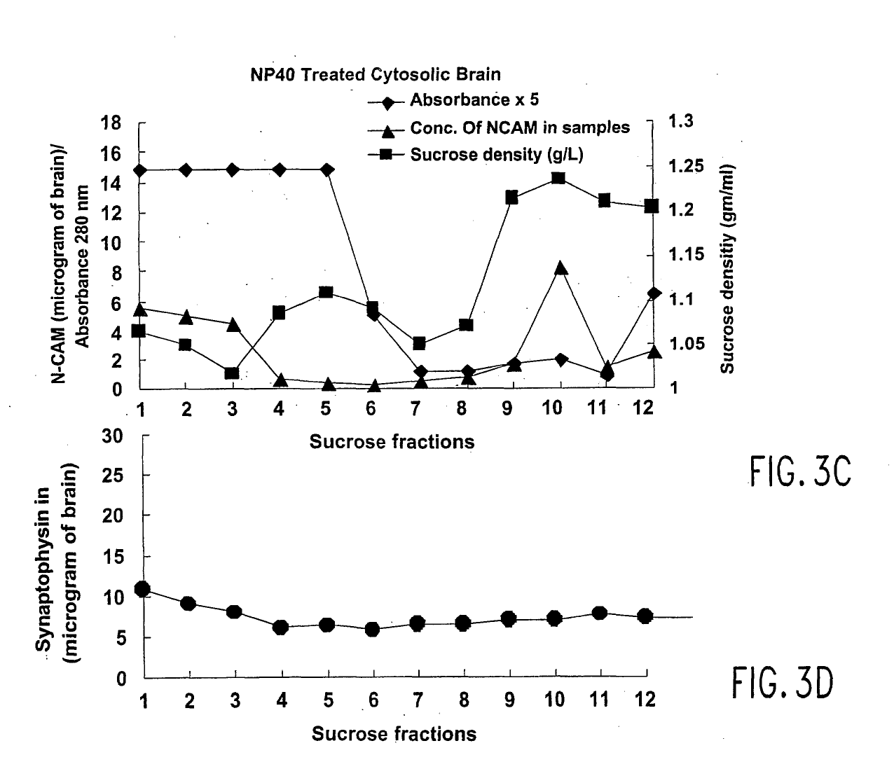

High density particles from sucrose density ultracentrifugation of cytosolic brain were obtained at a sucrose density of 1.194 g/L (FIG. 3A) and showed N-CAM immunoreactivity. The step gradient also showed synaptophysin immunoreactivity that co-migrated with cN-CAM (FIG. 3B). Pretreatment of the brain cytosol with NP-40 prior to ultracentrifugation altered the migration of N-CAM slightly in sucrose density gradient (FIG. 3C). Approximately 50% of cN-CAM stayed at the top of the gradient following NP-40 pretreatment and the remaining sedimented to the 1.237 g/L density. The NP-40 pretreatment disrupted the migration of synaptophysin in the sucrose gradient (FIG. 3D) so that synaptophysin was seen in low concentration in all sucrose fractions, due to membrane fragmentation.

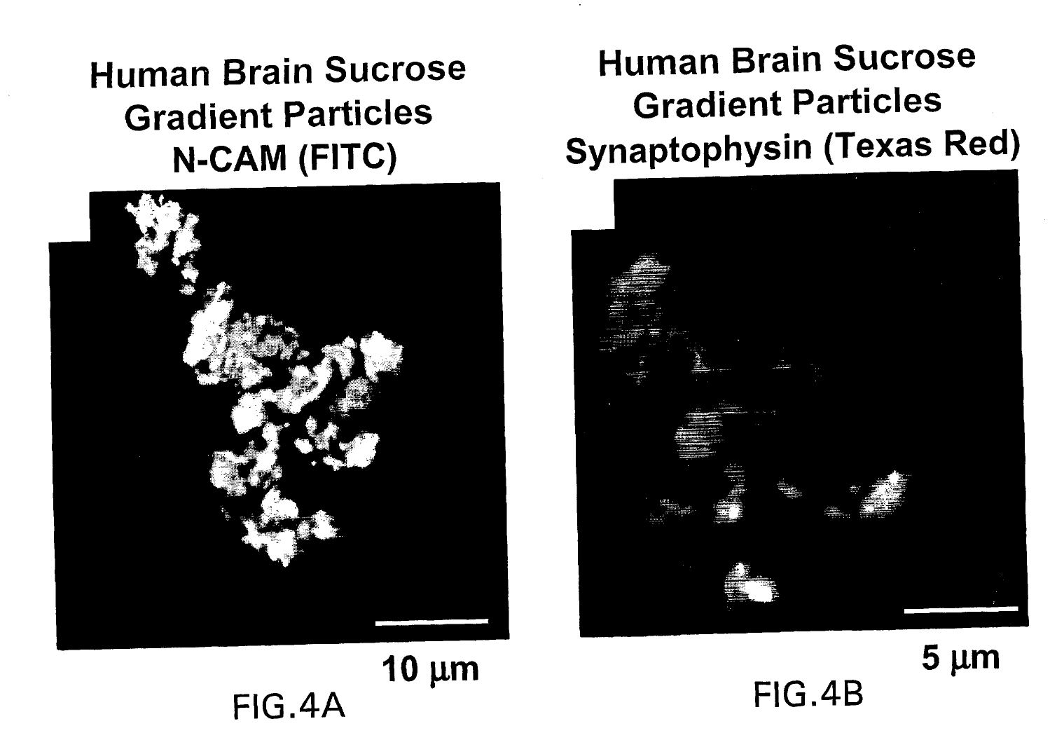

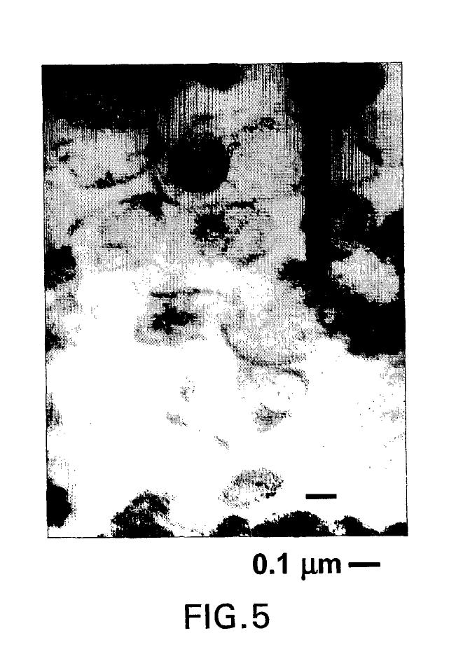

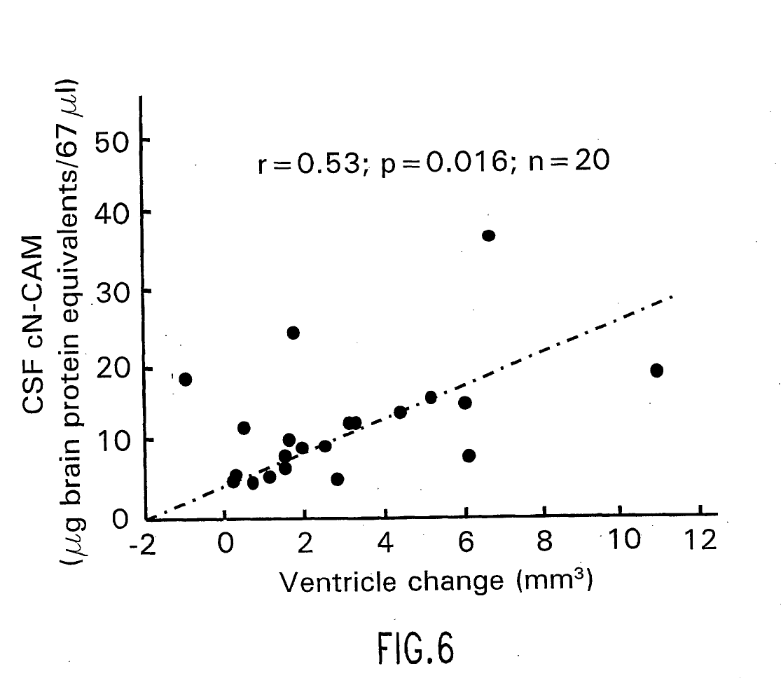

The control fraction from the brain sucrose density preparations PBS treatment only) was visualized by fluorescent immunocytochemistry for N-CAM (FIG. 4A) and synaptophysin (FIG. 4B). Electron microscopy results show irregular clusters of densely packed aggregated spheres and membranous particles (FIG. 5).

### EXAMPLE 4

cN-CAM is Strongly Related to Progressive Ventricular Enlargement in Patients With Schizophrenia.

CSF samples from patients with schizophrenia (n=20) were obtained by lumbar puncture in the am before breakfast following overnight food restriction. All patients met the diagnostic criteria for schizophrenia using DSM-III-R criteria⁴.

All subjects gave informed, written consent for participation in this study, which was approved by the Dallas VAMC Committee on Human Subjects. Patients were admitted for the study and were maintained on a standard hospital diet. Patients receiving neuroleptic drugs at the time of admission were “washed out,” i.e., were neuroleptic drug free, for at least 10 days prior to the lumbar CSF tap. The tap was performed between 7:30 and 8:00 AM following a 12 hour period of fasting and bed rest. Lorazepam (up to 4 mg/day) which often aided the patient during the neuroleptic free period was withheld for 10.5 hours prior to the lumbar tap. CSF was collected in the lateral decubitus position in 1 ml aliquots, which were immediately (at bedside) immersed in dry ice, with subsequent maintenance at −70° C. (or in dry ice during shipping) until the time of assay. The eighth 1 ml sample of CSF was used for the assessment of CSF N-CAM. The results showed a decrease in CSF N-CAM in first episode patients⁵. However, the patients were also given a Magnetic Resonance Imaging brain scan and then followed up for a period of 2 years and underwent a second scan using the same methodology⁶. The results show that N-CAM is associated at the time of lumbar puncture with the rate of change of brain ventricular size (r=0.53; p<0.016, n=20 patients undergoing N-CAM measurements and 2 repeat CT brain scans).

### EXAMPLE 5

Modulation of Polysialic Acid on N-CAM Decreases Protease Resistance to Breakdown.

Membrane “extracts” and “cytosolic” tissue extracts of human occipital cortex were prepared as described⁸ with slight modifications⁹. Frozen pulverized occipital cortex ˜50 g was suspended in 1000 ml of cold 0.05M Tris buffered saline (TBS, pH 7.4) with protease inhibitors: antipain (4 μg/ml), pepstatin A (2 μg/ml), aprotinin (2 μg/ml), leupeptin (2 μg/ml), and phenyl methyl sulfonyl fluoride (0.1 μg/ml). The solution was homogenized (Tissumizer; Tekmar, Cincinnati Ohio) for 5×10 sec pulses in a 4° C. ice bath and 30 sec cooling interval between pulses. The homogenate was centrifuged for 30 min at 42,000×g at 4° C. The clear supernatant with visible lipid removed was the “cytosolic” fraction. The pellet was resuspended and washed with cold TBS-protease inhibitor cocktail and re-centrifuged at 42,000×g for 30 min at 4° C. The supernatant was discarded and the pellet extracted in cold TBS+ protease inhibitor+1% NP-40 detergent. The solution was stirred at 4° C. for 30 min and then centrifuged at 42,000×g for 30 min. The supernatant was labeled “membrane extract.” Protein content of the “cytosolic” and “membrane” sample was measured by the micro bicinchoninic acid method (Pierce, Ill.).

Neuraminidase treatment of cytosolic and membrane brain extracts was accomplished by using neuraminidase enzyme from Vibrio cholerae (˜2.49 Units/ml, Fluka) in pH 5.5, 0.15M NaCl, 4 mM CaCl₂ that was diluted with 50 mM sodium acetate, 4 mM CaCl₂ and 0.2 mM EDTA, (pH 5.0; Buffer A) to 1.25 U/ml. Aliquots of cytosolic or membrane extracts of brain (100 μl) were mixed with 100 μl of diluted neuraminidase and incubated for 18 hrs at 37° C. Control aliquots of cytosolic and membrane extracts were treated with 100 μl of Buffer A. Additional controls were refrozen for 18 hrs. Samples were analyzed by immunoblot for N-CAM with antibody 3732.

Western Immunoblot Analyses

The procedure used previously for detection of N-CAM, synaptophysin, and actin was used as detailed⁹. Briefly, the cytosolic and membrane extracted fractions were diluted (1 volume sample: 1 volume loading buffer, SepraSol, Integrated Separation Systems, Natick, Mass.), denatured at 95° C. and 40-80 μg of protein separated by 7.5% SDS-PAGE¹⁰. The immunoblots were probed with N-CAM antisera, actin, or synaptophysin. Primary antibody binding to the blotted membrane was visualized with a secondary antibody (goat anti-rabbit IgG horseradish peroxidase conjugate, Sigma, St. Louis, Mo.) diluted 1:5,000 and an enhanced chemiluminescent reaction (Amersham Phannacia Biotech, Inc., Piscataway, N.J.). The membranes were exposed to BioMax MR film for 60 sec-300 sec (Kodak). The films were developed and images transferred by a flatbed scanner with 600 dpi resolution (HPScanJet) to a computer. The optical density for each band was measured with NIH Image software (v 1.59b, Wayne Rasband, NIH).

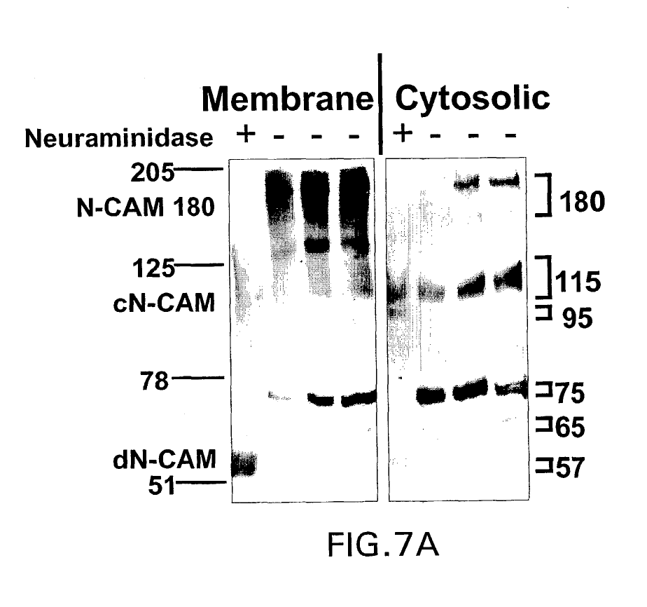

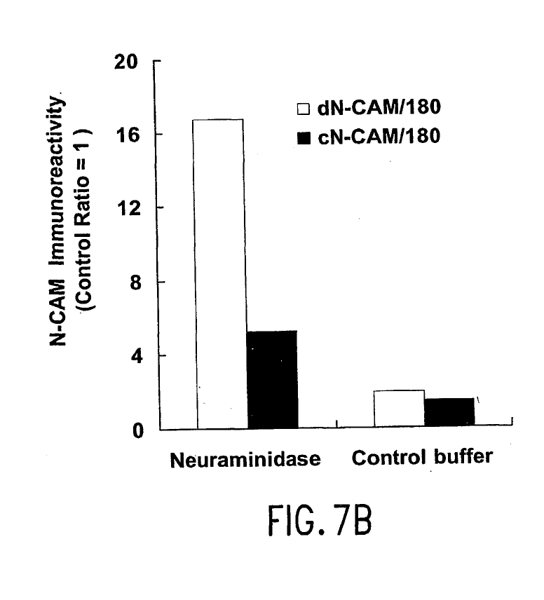

Neuraminidase treatment produces a minor alteration of cN-CAM resulting in a faster migrating immunoreactive band indicating partial glycosylation of cN-CAM (FIG. 7A), while neuraminidase incubation with membrane extracts resulted in removal of higher MW N-CAM 180. This result in which we might expect a preservation of N-CAM 180 and only a minor modification from adult brain, indicates that upon removal of the polysialic acid residues in N-CAM 180 parent molecules the resistance to protease breakdown is lost. Although, we observe cN-CAM as expected from breakdown of the N-CAM 180 there is only a partial conversion to cN-CAM with the majority converted to a novel dN-CAM (≈51 kDa peptide). Thus, N-CAM 180 appears to degrade via endogenous neuraminidase to N-CAM 105-115, but upon first removing polysialic acid the breakdown continues to a fragment now identified as dN-CAM (≈51 kDa peptide). The conversion product of N-CAM 180 to N-CAM 105-115 is found in higher abundance in neuraminidase treated cytosolic extracts of brain, while the breakdown of N-CAM 180 via removal of polysialic acid and subsequent breakdown to dN-CAM (≈51 kDa peptide) occurs largely in membrane bound N-CAM isoforms (FIG. 7B).

### EXAMPLE 6

Specific Assay for Monitoring the Conversion of Parent Molecule N-CAM to cN-CAM in Human Brain.

Fresh or frozen human brain cortex is dissected free of pia mater layer and any obvious blood vessels on the surface of the cortex. Pulverize the cortex in a sufficient quantity of liquid Nitrogen to a friable state with mortar and pestle, and accurately weigh out replicate samples of pulverized brain sample while keeping all vessels frozen in dry ice. Place the quantity of protease inhibitor or neuraminidase inhibitor in a PBS buffer of final pH 7.4 and maintain buffer on ice. Add the varying concentrations and combinations of protease inhibitors and/or neuraminidase inhibitors to separate pulverized brain in tubes in a final volume to yield a 20% weight of brain tissue/buffer volume. Allow the tubes to adjust to room temperature, and incubate at standard room temperature conditions for varying time points. The end-point of the assay is accomplished by addition of a 2× denaturing reducing sample buffer such as commercially available and previously published, and heat 3 min at 95 degrees C. Proceed to standard SDS-PAGE and immunoblot conditions to determine the breakdown products of parent molecule N-CAM. Use appropriate control incubations for temperature breakdown and buffer breakdown without protease inhibitors and/or neuraminidase inhibitors. The results can be quantitatively assessed by densitometry of immunoblots as previously published which yields a linear response within a loading range of proteins (˜1-100 μg of protein) depending on gel capacity used and concentrations of antibody and detection substrates which are empirically determined with standard methods. This method has not been published for human brain and is novel and useful in determining whether compounds are capable of effectively modulating enzymes present within the complex environment of human brain. See FIGS. 2A and 2B. The use of a human cell culture with this method does not yield similar results as there is a lack of the enriched connections and extracellular matrix molecule (ECM) present in brain tissue.

Use fresh or frozen human brain synaptosomes or neurosynaptosomes or neurosomes.

The modulation of N-CAM fragments occurs in synaptosomes in the brain. The assay used above for screening protease inhibitors can be applied to the synaptosomal preparation from human brain as a model system for determining partial therapeutic efficacy in preclinical trials of neuraminidase inhibitors and protease inhibitors for treatment of the neuropsychiatric diseases described herein. Further, as described below, in vivo monitoring of therapeutic effect can be made by monitoring breakdown fragments of N-CAM in CSF. See FIGS. 1A and 1B.

### EXAMPLE 7

Treatment of Patients with Protease Inhibitor and Neuraminidase Inhibitor

A reduction in the progression of chronic schizophrenia is demonstrated with serine-protease inhibitors, aspartic-protease inhibitors, cysteine-protease inhibitors, aminopeptidase-protease inhibitors and neuraminidase inhibitors which are compounds shown to reduce N-CAM breakdown in vivo and in vitro. Breakdown products of N-CAM in humans have been shown to correlate with positive symptoms (psychosis) and ventricular enlargement (accelerated brain aging) in patients with schizophrenia. Serine protease inhibitors reduce the breakdown of N-CAM in rodent brain¹¹. Patients are afforded the opportunity to halt and reverse the progressive features of schizophrenia: positive symptom (psychosis), ventricular enlargement (accelerated brain aging), and cognitive decline in a controlled clinical trial.

The standard of care for patients with schizophrenia consists of pharmacotherapy with “typical” and “atypical” neuroleptics. These compounds to date have not been shown to be effective in eliminating the positive symptoms of schizophrenia, slowing or reversing the neurocognitive decline, halting progressive ventricular enlargement, or returning CSF accumulation of N-CAM breakdown products to normal levels. Thus, many patients with schizophrenia and the diseases described herein who receive standard therapy benefit from a regimen of additional treatment to manage the positive symptoms of schizophrenia, slow the characteristic neurocognitive deficits or reverse the neurocognitive decline, slow the progressive ventricular enlargement, and lower the CSF concentrations of N-CAM breakdown products to a normal level.

Therapeutically appropriate dosages for (A) aspartic protease inhibitors, (B) serine protease inhibitors, (C) cysteine protease inhibitors, (D) aminopeptidase inhibitors and (E) neuraminidase inhibitors are listed in Tables 2, 3 and 4. The compounds or combinations of compounds from these classes of compounds (A, B, C, D and E) are administered in dosages that are effective in reducing the breakdown products of N-CAM in CSF as measured prior to treatment and during treatment.

Reductions in CSF N-CAM breakdown products are correlated with improvements in cognitive functioning (4 weeks and later), positive and negative symptom reduction (4 weeks and later), and longer term MRI changes in CSF ventricular space at the two year clinical trial time point.

The present treatment is applicable to any patient, usually between the ages of 18 and 65 years old who has a history of a Psychotic Disorder, NOS; Schizophrenia; Schizoaffective Disorders; or other Neuropsychiatric Disorders as defined herein.

The patient will preferably be abstinent from alcohol and all illicit drugs for at least 30 days at the time of initial lumbar puncture and 16 week lumbar puncture.

If needed, a patient can be stabilized on a “typical” or “atypical” neuroleptic treatment regimen for 30 days prior to treatment according to the present invention. If the patient has never been treated with neuroleptics, under the discretion of medical responsible psychiatrist, neuroleptics may be prescribed as part of the present protocol.

The patient should exhibit no uncorrectable loss of hearing or eyesight that precludes psychometric testing and should have the ability to comprehend instructions or respond to test items of the Repeatable Battery for the Assessment of Dementia (RBAD) during baseline administration and Mini Mental Status Examination.

Treatment Side Effects

Aprotinin data from clinical trials indicate that it is generally well tolerated in humans,² with few adverse events. Hypersensitivity reactions occur in <0.1 to 0.6% of patients receiving aprotinin for the first time. Clinical evidence to date supports the use of aprotinin over its competitors in patients at high risk of hemorrhage, in those for whom transfusion is unavailable or in patients who refuse allogeneic transfusions.

Amprenavir, as with other protease inhibitors, may be associated with acute hemolytic anemia, diabetes mellitus, and hyperglycemia, but the drug's effects on patients lipid profiles at this point appears clinically insignificant. The most frequently reported adverse events in clinical trials were nausea, diarrhea, vomiting, rash, and perioral paresthesia. Severe and life-threatening skin reactions, including Stevens-Johnson syndrome, occurred in 1% of patients treated with amprenavir. Amprenavir is taken twice a day, with or without food, but it should not be taken with a high-fat real, as that would decrease the absorption of the drug. The most common side effects are gastrointestinal (nausea, vomiting, and/or diarrhea), rashes, and oral paresthesia (a tingling sensation around the mouth). Patients should know that severe or life-threatening rash has occurred is 1% of recipients (4% of those who develop a rash), and that “amprenavir therapy should be discontinued for severe or life-threatening rashes and for moderate rashes accompanied by systemic symptoms” (Physician's Desk Reference). Pregnant women should not use amprenavir unless it is medically necessary, because of concern about harm to the fetus in some animal tests (there are no human data). Also, several prescription drugs must not be taken with amprenavir, and several others require blood tests to monitor drug levels. Patients are monitored for these and other similar adverse reactions by the physician or other health care provider.

Drug Route, Doses, Frequency, Duration

Oral, inhalant, nasal spray. Doses are adjusted to within IND and FDA recommended levels (e.g., see Tables 2, 3, 4 and 5) in an ascending phase for 2 weeks prior to achieving therapeutic dosage levels. Patients receive a protease inhibitor or a neuraminidase inhibitor or a combination of a protease inhibitor and a neuraminidase inhibitor in a safely administered dosage according to physician recommendation.

Lumbar Tap (Puncture) Procedure

About 10 ml of CSF is removed for study from the lumbar region by needle, and the patient is given a local anesthetic prior to the lumbar tap. The procedure lasts between 5-15 minutes, and the patient is restricted to a short rest and observation prior to resuming ambulatory activities.

Lumbar taps can cause headaches in 30-40% of patients within the first few days after a lumbar puncture. Usually the headaches disappear without treatment beyond a mild pain reliever. Prolonged headaches, lasting longer than seven day, occur in about 0.5 to 2 percent of patients. These prolonged headaches usually taper off within two weeks of the lumbar puncture. For prolonged headache, a blood patch, that is a small injection of blood into the area of the back where the lumbar puncture was performed is sufficient to seal any CSF leak and cause the headaches to disappear. The only other risk to CSF lumbar tap is temporary double vision and infection.

Magnetic Resonance Imaging of the Brain

Magnetic Resonance Imaging (MRI) of the brain uses a magnetic field and radio waves and is more sensitive to structural changes than X-ray and carries no radiation risk. A patient is placed in a cylinder for up to one hour with monitoring and asked to remain still for 10-15 minutes at a time.

Evaluations prior to drug administration can include the following:

Brain MRI scan, CSF lumbar tap (10 ml), Neuropsychological instruments to include Repeatable Battery for Assessment of Dementia, Mini Mental Status Exam, Wechsler Memory Scale, Wisconsin Card Sort, Trails A and Trails B, Finger Tapping, Rey Auditory Verbal Learning Test, Olfactory Identification Test, Subtests of the Wechsler Adult Intelligence Scale (Digit Symbol, Block Design, Arithmetic, Similarities, Vocabulary), and the Wide Range Achievement Test—Reading, as detailed in Handbook of Neuropsychological Testing Muriel Lezak). Psychiatric symptom assessment scale (Scale for Assessment of Negative Symptoms (SANS) and Scale for Assessment of Positive Symptoms (SAPS) by Nancy Andreasen, Univ. of Iowa.

After treatment the measures of positive and negative symptoms and neuropsychological scores adjusted for age norms are evaluated by separate one way ANOVAs to determine if treatment produces an improvement that is significant.

Evaluations after 4 and 8 weeks of drug administration can include the following:

Repeatable Battery for Assessment of Dementia, Mini Mental Status Exam, Finger Tapping, Olfactory Identification Test, Digit Symbol, Block Design, Scale for Assessment of Negative Symptoms (SANS) and Scale for Assessment of Positive Symptoms (SAPS).

Evaluations after 12 weeks of drug administration can include the following:

CSF lumbar tap, optional (10 ml)

Evaluations after 16 weeks of drug administration can include the following:

Neuropsychological instruments to include Repeatable Battery for Assessment of Dementia, Mini Mental Status Exam, Wechsler Memory Scale, Wisconsin Card Sort, Trails A and Trails B, Finger Tapping, Rey Auditory Verbal Learning Test, Olfactory Identification Test, Subtests of the Wechsler Adult Intelligence Scale (Digit Symbol, Block Design, Arithmetic, Similarities, Vocabulary), and the Wide Range Achievement Test—Reading. Psychiatric symptom assessment scale (Scale for Assessment of Negative Symptoms (SANS) and Scale for Assessment of Positive Symptoms (SAPS).

Evaluations after 52 weeks of drug administration can include the following:

(optional) CSF lumbar tap, 10 ml

(optional) MRI scan

Evaluations after 104 weeks of drug administration can include the following:

(optional) CSF lumbar tap, 10 ml

(optional) MRI scan

### EXAMPLE 8

Occipital cortex obtained from squirrel monkey (genus Saimirri) was extracted by the same method for cytosolic and membrane fractions of human brain described herein, and for example, see Example 9.

To confirm the efficacy of protease inhibitors in reducing buildup in the brain of cNCAM or other breakdown products of N-CAM, a squirrel monkey is injected with the protease inhibitor intravenously (dosing as shown in Table 4). N-CAM, c-N-CAM or other fragments of N-CAM can be measured in CSF pre- and post-injection of protease inhibitor to determine the degree of inhibition of release of cN-CAM, N-CAM or other fragments into CSF.

### EXAMPLE 9

Membrane and cytosolic fractions from human occipital cortex were incubated in the presence of protease inhibitors (10 μM final concentration) for varying lengths of time (typically 2, 4, 8, 16, 32, and 48 hrs at room temperature). An aliquot was analyzed by gel electrophoresis and immunoblotted with N-CAM 3732 antibody. Protease inhibitors that were assayed individually were: Aprotinin, AEBSF, Antipain, Bestatin, Amastatin, Elastatinal, AMPSF, Leupeptin, Pepstatin A, PMSF, L-trans-epoxysuccinyl-leucylamide-(4-guanido)-butane, and Amastatin ([(2S, 2R)]-3-Amino-2-hydroxy-5-methylhexanoyl]-Val-Val-Asp-OH). All protease inhibitors were obtained from Sigma. The protease inhibitors that showed the most activity in prevention of the breakdown of N-CAM 180 and subsequent release of cN-CAM were leupeptin (FIG. 8B) and antipain (FIG. 8C). To prevent breakdown of N-CAM during extraction of cytosolic and membrane fractions, the following standard protease inhibitor cocktail was utilized: antipain (4 μg/ml), pepstatin A (2 μg/ml), aprotinin (2 μg/ml), leupeptin (2 μg/ml), and phenyl methyl sulfonyl fluoride (0.1 μg/ml). The brain tissue was placed in the protease inhibitor solution and homogenized (Tissumizer; Tekmar, Cincinnati Ohio) for 5×10 sec pulses in a 4° C. ice bath with a 30 sec cooling interval between pulses. The homogenate was centrifuged for 30 min at 42,000×g at 4° C. The clear supernatant with visible lipid removed was the “cytosolic” fraction. The pellet was resuspended and washed with cold TBS-protease inhibitor cocktail and re-centrifuged at 42,000×g for 30 min at 4° C. The supernatant was discarded and the pellet extracted in cold TBS+ protease inhibitor+1% NP-40 detergent. The solution was stirred at 4° C. for 30 min and then centrifuged at 42,000×g for 30 min. The supernatant was labeled “membrane extract.” Protein content of the “cytosolic” and “membrane extract” sample was measured by the micro bicinchoninic acid method (Pierce, Ill.). FIG. 8A shows the standard protease inhibitor cocktail effects, FIG. 8B shows the addition of leupeptin (10 μM concentration) and FIG. 8C shows the addition of antipain to a 10 μM concentration.

### EXAMPLE 10

Measurement of neuroserpin gene by microarray in patients with schizophrenia was used to screen 1128 human genes relevant to brain function. Two brain regions were investigated using pools of total RNA from patients with schizophrenia and controls: dorsolateral prefrontal cortex (Brodmann area (BA) 46 and prefrontal cortex (PFC), (BA)9). A neuroarray was developed and the details of the development of the NIA-Neuroarray are available at URL http://www.grc.nia.nih.gov/branches/rrb/dna.htm) and are reported (Vawter, et al., Brain Research Bulletin, 2001). Briefly, a 15,000 human cDNA clone set of IMAGE Consortium clones (http://image.llnl.gov)/) available from Research Genetics (Huntsville, Ala.) was sorted for brain relevant genes. Also, Medline™ was consulted for relevant reports of protein or MRNA screening of patients with neuropsychiatric disorders. This resulted in a list of genes (1128 clones) representing families such as transcription factors, synaptic, neuronal, glial, cell adhesion molecules, kinases, phosphatases, proteases, oncogenes, and structural genes that were chosen for inclusion in the neuroarray. The Stanley Foundation provided a set of cerebellum and prefrontal cortex samples from drug treated patients with schizophrenia (n=5) and controls (n=5) that were age matched. A second cohort of tissue samples from the PFC (BA 46) were provided by the Clinical Brain Disorders Branch at NIMH and consisted of patients with schizophrenia (n=15) and controls (n=15). The 15 samples of the PFC obtained from controls formed 3 pools of 5 samples and similarly 3 matched pools of patients with schizophrenia were formed. In total, 4 pools of patients with schizophrenia (n=20) and 4 pools of controls (n=20) were analyzed. The total RNA was extracted from each brain sample by first pulverizing the frozen tissue in liquid N₂ with a mortar and pestle to a fine powder. About 0.1-0.2 g of the brain powder was homogenized in ice-cold Trizol (Life Technologies Inc, Rockville Md.) using a Tissumizer (Tekmar, Cincinnati, Ohio) at #40 speed for 3×30 sec pulses and 30 sec cooling. The samples were processed by the guanidinium thiocyanate method (Chomczynski and Sacchi, 1987) according to the procedure recommended for Trizol extraction, i.e. 0.2 vol chloroform was added to the Trizol-brain homogenate, samples hand shaken for 30 see vigorously, and centrifuged at 12,000×g for 20 min at 4° C. The supernatant was transferred to a fresh tube and the RNA precipitated with 0.5 vol isopropyl alcohol, centrifuged as above, and the pellet was extracted with 75% ethanol. The ethanol mixture was centrifuged at 7,500×g for 20 min at 4° C. The supernatant was decanted and the pellet was resuspended in 75% ethanol, and recentrifuged at 10,000×g for 10 min at 4° C. The resulting pellet was left slightly wet and partially dried at room temperature under vacuum. The pellet was resuspended in TE buffer, pH 7.5 and a 1:100 dilution was made in DEPC -treated H₂0, and the A₂₆₀ and A₂₆₀/A₂₈₀ ratio obtained in a Beckman spectrophotometer (DU-64, Fullerton Calif.). The yield of total RNA was in the range between 20-90 μg per 100 mg of brain tissue for all brain regions. A sample of the total RNA was diluted to 1 μg/μl with a 10× RNA sample buffer (Quality Biologicals, Gaithersburg Md.) and run on a denaturing 1.2% formaldehyde agarose gel. The resulting 28S and 18S ribosomal bands were visualized as well as any streaking of DNA or degradation of samples. The best quality samples judged by gross examination of the gels were used for pools. Further the A₂₆₀/A₂₈₀ ratio was usually >1.9 or the sample was considered for re-extraction. A pool of total RNA (20 μg) was formed for each group using equal amounts of total RNA from each individual.

RNA Labeling and Hybridization

The procedure described for radioactive labeling of total RNA with ^{[33]-}-P-dCTP was followed (URL http://www.grc.nia.nih.gov/branches/rrb/dna.htm). Briefly, total RNA (20 μg) is reverse transcribed to cDNA with reverse transcriptase enzyme in the presence of ^{[33]-}P-dCTP. The ^{[33]-}P-dCTP-cDNA is purified through a spin column by size separation (BioSpin, Bio-Rad, CA) from ^{[33]-}P-dCTP and the heat denatured probe (˜5×10⁶ cpm) is diluted in 4 ml of Microhyb solution (Research Genetics) and hybridized to the neuroarray for 16-18 h at 50° C. with rotation. Two washes with 2× SSC at room temperature are carried out to remove unhybridized probe. The neuroarray is placed under saran wrap and exposed to a low energy phosphor screen (Molecular Dynamics, Sunnyvale, Calif.) for 1-5 days and scanned in a Phosphorimager 860 (Molecular Dynamics) at 50 μm resolution.

A z-score normalization method was devised and applied to each hybridization image. This method involves calculating a distribution of z-scores for all genes in each array, and employing the differences of the z-scores (z-ratio) between the two conditions to search for genes for which expression is changed in schizophrenia. The mean and the standard deviation of the log₁₀ scores for each pool are calculated and entered into a z-score normalization formula: Observed Gene z-score=(Observed Gene log₁₀ intensity−Mean Neuroarray Pool log₁₀ intensity)/(standard deviation Neuroarray Pool log₁₀ intensity). Gene expression differences between two neuroarrays, i.e., schizophrenia and control neuroarrays, are calculated by taking the difference between observed gene z scores=[(z_{S1a+}z_{S1b})/2]−[(z_{C1a+}Z_{C1b})/2] where S1 and C1=schizophrenia gene 1 and control gene 1, respectively and a, b, represent individual z-scores obtained from 2 measurements of the gene. The mean z-score difference for all genes on two neuroarrays (S-C) is 0; however, the standard deviation of the z differences distribution ranges between ˜0.2-0.4 for each neuroarray. To facilitate comparison to traditional fold differences, the z-score differences are further translated into z-ratios based upon the formula: z-ratio= (z score difference gene 1/standard deviation of the z differences distribution).

By using the above method, it was shown that in 4 pools of patients with schizophrenia, the neuroserpin gene has the largest average down-regulation of gene expression among all 1128 genes surveyed (FIG. 2). Neuroserpin is an endogenous serine protease inhibitor. This deficiency in expression of the gene for an endogenous serine protease inhibitor in the PFC of patients with schizophrenia can be responsible for elevation in cN-CAM. The effect is most noticeable in pools of patients that have the greatest male composition, in that pool 3 has the fewest male patients and showed the smallest change in neuroserpin.

### REFERENCES

1. Docagne, F., Nicole, O., Marti, H. H., MacKenzie, E. T., Buisson, A., & Vivien, D. (1999). Transforming growth factor-beta 1 as a regulator of the serpins/t-PA axis in cerebral ischemia. Faseb J, 13(11):1315-24.

2. Martin, E. W. Remington's Pharmaceutical Sciences, Martin, latest edition, Mack Publishing Co., Easton, Pa. See Page 17.

3. Vawter, M. P., Howard, A. L., Hyde, T. M., Kleinman, J. E., & Freed, W. J. (1999). Alterations in SEC N-CAM in bipolar disorder and synaptophysin in schizophrenia. Molecular Psychiatry, 4: 467-475.

4. Kendler, K. S., Spitzer, R. L., & Williams, J. B., Psychotic disorders in DSM-III-R; Am. J. Psychiatry August 1989,: 146(8):953-62.

5. Vawter, M. P., Hemperly, J. J., Freed, W. J., & Garver, D. L. (1998); CSF N-CAM in neuroleptic-naive first-episode patients with schizophrenia; Schizophr Res, 34(3): 123-31.

6. Nair, T. R., Christensen, J. D., Kingsbury, S. J., Kumar, N. G., Terry, W. M., & Garver, D. L. (1997), Progression of cerebroventricular enlargement and the subtyping of schizophrenia, Psychiatry Res, 74(3), 141-50.

7. Vawter, M. P., Hemperly, J. J., Hyde, T. M., Bachus, S. E., VanderPutten, D. M., Howard, A. L., Cannon-Spoor, H. E., McCoy, M. T., Webster, M. J., Kleinman, J. E., & Freed, W. J. (1998). VASE-containing N-CAM isoforms are increased in the hippocampus in bipolar disorder but not schizophrenia. Exp Neurol, 154(1), 1-11.

8. Takamatsu, K., B. Auerbach, R. Gerardy-Schahn, G. Jaques, D. Krause, & N. Madry (1994), Evaluation of the binding properties of monoclonal antibodies submitted to Third International IASLC Workshop on Lung Tumor and Differentiation Antigens to Neural Cell Adhesion Molecule, Int. J. Cancer Suppl., 8:30-33.

9. Vawter, M. P., Cannon-Spoor, H. E., Hemperly, J. J., Hyde, T. M., VanderPutten, D. M., Kleinman, J. E., & Freed, W. J. (1998), Abnormal expression of cell recognition molecules in schizophrenia, Experimental Neurology, 149(2):424-432.

10. Hochstrasser, D. F., Harrington, M. G., Hochstrasser, A. C., Miller, M. J., & Merril, C. R. (1988), Methods for increasing the resolution of two-dimensional protein electrophoresis, Anal Biochem, 173(2):424-35.

11. Endo, A., Nagai, N., Urano, T., Ihara, H., Takada, Y., Hashimoto, K., & Takada, A. (1998). Proteolysis of highly polysialylated NCAM by the tissue plasminogen activator-plasmin system in rats. Neurosci Lett, 246(1), 37-40.

12. Peters, D. C., Noble S. (Feb. 1999) Drugs, 57(2):233-60.

13. Chomczynski, P., Sacchi, N. (1987): Single-step method of RNA isolation by acid guanidinium thiocyanate-phenol-chloroform extraction. Anal Biochem, 162:156-159.

14. Duggan, D. J., Bittner, M., Chen, Y., Meltzer, P., Trent, J. M. (1999): Expression profiling using cDNA microarrays. Nat Genet, 21: 10-14.

15. Vawter, M. P., Barrett, T., Cheadle, C. Sokolov, B. P., Wood, W. H., Donovan, D. M., Webster, M., Freed, W. J., Becker, K. G. (2001). Application of cDNA microarrays to examine gene expression differences in schizophrenia. Brain Research Bulletin.

16. Whitney, L. W., Becker, K. G., Tresser, N. J., Caballero-Ramos, C. I., Munson, P. J., Prabhu, V. V., Trent, J. M., McFarland, H. F., Biddison, W. E. (1999): Analysis of gene expression in multiple sclerosis lesions using cDNA microarrays. Ann Neurol, 46: 425-428.

TABLE 1

| | |
|---|---|
| FDA Approved Inhibitors and | |
| Neuraminidase Inhibitors | Not FDA approved |

Aspartic Protease Inhibitors (A)

| | | | | | | |
|---|---|---|---|---|---|---|
| Nelfinavir | Saquinavir | Indinavir | Amprenavir | Ritonavir | Pepstatin | AG1776* |
| (Viracept) | (Invirase) | (Crixivan) | (Agenerase) | (Norvir) | | |
| | (Fortovase) | | | | ABT-387* | Beta- |
| | | | | | | secretase |
| | | | | | | inhibitors |

Serine Protease Inhibitors (B)

| | | |
|---|---|---|
| Aprotinin | AEBSF | Serpins |
| | Leupeptin | Autipain |
| | Elastatinal | APMSF |
| | | PMSF |

Cysteine Protease Inhibitor (C)

| | |
|---|---|
| AG7088* | Leupeptin |
| E-64 | |

Aminopeptidase Inhibitor (D)

| | |
|---|---|
| Bestatin* | Amastatin |
| (ubenimex) | |

Neuraminidase Inhibitors (E)

| | | |
|---|---|---|
| Zanamivir | RWJ- | GS 4071* |
| | 270201* | |
| Oseltamivir | GS 4104* | |
| | or RO 64- | |
| | 0797* | |

*In clinical trials testing.
Protease inhibitor cocktail component

TABLE 2
Protease Inhibitors and Neuraminidase Inhibitors

| | | | | | | Daily | Daily |
|---|---|---|---|---|---|---|---|
| | | | | | Administration | Dosage | Optimal |
| Drug | Class | Dosage | | Times/day | Route | Range | Dose |
| FDA | | | | | | | |
| Approved | | | | | | | |
| Nelfmavir | A | 750 | mg | 3 | Oral | 1000 mg- | 1500 mg |
| (Viracept) | | 1250 | mg | 2 | Oral | 2500 | |
| | | | | | | mg | |
| Saquinavir | A | 300 | mg | 4 | Oral | 1200 mg- | 1500 mg |
| (Invirase) | | 600 | mg | 3 | Oral | 1800 | |
| (Fortovase) | | 1200 | mg | 1 | Oral capsule | mg | |
| | | 1600 | mg | 1 | Oral capsule | | |
| | | 1800 | mg | 1 | Oral capsule | | |
| | | 600 | mg | 3 | Oral | | |
| Indinavir | A | 600 | mg | 3 | Oral | 1800 mg | 1800 mg |
| (Crixivan) | | | | | | | |
| Amprenavir | A | 1200 | mg | 2 | Oral | 2400 mg | 2400 mg |
| (Agenerase) | | | | | | | |
| Ritanavir | A | 100 | mg | 1-2 | Oral | 100- | 150 mg |
| (Norvir) | | 6 | capsules | 2 | Oral | 200 mg | |
| | | 7.5 | mL | 2 | Oral | | |
| Zanamivir | E | 12.8 | mg | 1 | intranasal | 7.2- | 600 mg |
| | | 10 | mg | 2 | inhalant | 1200 mg | (can be |
| | | 20 | mg | 2 | inhalant | | given |
| | | 64 | mg | 1 | inhalant | | orally) |
| | | 600 | mg | 2 | intravenously | | |
| | | 1200 | mg | 1 | intravenously | | |
| | | 3.6-16 | mg | 2-6 | intravenously | | |
| Oseltamivir | E | 75 | mg | 1-2 | Oral | 150 mg | 150 mg |
| Aprotinin | B | 140 | mg | 1-7 | Intravenous | 140- | 560 mg |
| | | | | | infusion | 980 mg | |

TABLE 3

| | | | | | Daily | Daily |
|---|---|---|---|---|---|---|
| | | | | Administration | Dosage | Optimal |
| Drug | Class | Dosage | Times/day | route | Range | Dose |
| IND | A | 133.33 mg | 6 | Oral | 200- | 500 mg |
| ABT-378* | | 200 mg | 1-2 | | 800 mg | |
| | | 400 mg | 2 | | | |
| | | 600 mg | 1 | | | |
| | | 800 mg | 1 | | | |
| AG7088*- | C | dosing not | | Oral | 10 mg- | |
| | | available | | | 1000 mg | |
| Bestatin* | D | 10 | 1 | Oral | 10-180 | 100 mg |
| (ubenimex) | | 30 | 1 | | mg | |
| | | 90 | 1 | | | |
| | | 180 | 1 | | | |
| GS 4071* | E | 75 mg | 2 | Oral | 150 mg | 150 mg |
| GS 4104* | E | 1-2 | 150 mg | Oral | 150 mg | 150 mg |
| or RO 64- | | | | | | |
| 0797*- | | | | | | |
| RWJ- | E | 200 mg | 2 | Oral | 400 mg | 400 mg |
| 270201* | | 400 mg | 1 | | | |

TABLE 4
Projected Dosage and Routes, Pre-Clinical In Vivo Testing to Establish Route, Safety
and Tolerability. These are Prototype Compounds As Examples of inhibitors in
Pre-Clinical Usage and Study

| | | | | | Daily | Daily | |
|---|---|---|---|---|---|---|---|
| | | | | Administration | Dosage | Optimal | |
| Drug | Class | Dosage | Times/day | Route | Range | Dose | |
| Pre-clinical | | | | | | | |
| (Prototype | | | | | | | |
| compounds) | | | | | | | |
| and Classes | | | | | | | |
| AG1776 | A | 750 mg | 3 | Oral | 1000 mg- | 1500 | mg |
| | | 1250 mg | 2 | Oral | 2500 mg | | |
| Beta-secretase | A | Compound | | | | | |
| inhibitor class | | not | | | | | |
| | | available | | | | | |
| Pepstatin | A | 100 mg | 7 | Oral | 700 | 700 | mg |
| AEBSF | B | 50 mg | 4 | Oral | 200 mg | 200 | mg |
| Leupeptin | B | 100 mg | 4 | Oral | 400- | 1100 | mg |
| | | 900 mg | 2 | Oral | 1800 mg | | |
| Antipain | B | | | Oral | 30 mg- | 300 | mg |
| | | | | | 1500 mg | | |
| Elastatinal | B | | | Oral | 30 mg- | 150 | mg |
| | | | | | 300 mg | | |
| APMSF | B | | | Oral | 15 mg- | 150 | mg |
| | | | | | 1500 mg | | |
| PMSF | B | | | Oral | 0.5 mg- | 1.5 | mg |
| | | | | | 15 mg | | |
| Serpins | B | Compound | | | | | |
| | | not | | | | | |
| | | available | | | | | |
| E-64 | C | | | | 20- | 50 | mg |
| | | | | | 200 mg | | |
| Leupeptin | C | 100 mg | 4 | Oral | 400- | 1100 | mg |
| | | 900 mg | 2 | | 1800 mg | | |

TABLE 5

| | | | | |
|---|---|---|---|---|
| Amastatin | D | Oral | 50 mg- | 200 mg |
| | | | 500 mg | |
| — | E | | | 500 mg |
| trifluoroacet | | | | (Oral) |
| yl analogue, | | | | |
| of 4- | | | | |
| guanidino- | | | | |
| Neu5Ac2en | | | | |
| (FANA) | | | | |

Legend for Tables 1, 2, 3, 4 and 5
(A) - Aspartic protease inhibitors
(B) - Serine protease inhibitors
(C) - Cysteine protease inhibitor
(D) - Aminopeptidase inhibitor
(E) - Neuraminidase inhibitors
(IND) - Investigational new drug

TABLE 6

| | | |
|---|---|---|
| Time 0 (original sample) | Time 2 (4-8 hours) | Time 3 (48 hours) |
| SA1, SB1 (soluble with and | SA2, SB2 (soluble with and | SA3, SB3 (soluble with and |
| without protease inhibitor) | without protease inhibitor) | without protease inhibitor) |
| CA1, CB1 (crude with and | CA2, CB2 (crude with and | CA3, CB3 (crude with and |
| without protease inhibitor) | without protease inhibitor) | without protease inhibitor) |

What is claimed is:

1. A method of treating a neuropsychiatric disease in a human subject comprising administering a therapeutically effective amount of at least one compound selected from the group consisting of protease inhibitors and neuraminidase inhibitors, whereby administering the compound to the subject treats the neuropsychiatric disease.

2. The method of claim 1, further comprising administering a neuroleptic to the subject.

3. The method of claim 1, wherein a protease inhibitor inhibits a protease selected from the group consisting of serine proteases, metalloproteinases, aspartyl proteases, cysteine proteases and aminopeptidases.

4. The method of claim 1, wherein a neuraminidase inhibitor inhibits a neuraminidase selected from the group consisting of neuraminidase 1, neuraminidase 2 and neuraminidase 3.

5. The method of claim 1, wherein the protease inhibitor and the neuraminidase inhibitor are in a pharmaceutically acceptable carrier.

6. The method of claim 1, wherein the neuropsychiatric disease is selected from the group consisting of Schizophrenia, Bipolar Disorder I, Bipolar Disorder I with Psychotic Features, Bipolar Disorder II, Bipolar Disorder II with Psychotic Features, Psychotic Disorder Not Otherwise Specified, Schizophreniform Disorder, Schizoaffective Disorder, Unipolar Disorder, Unipolar Disorder with Psychotic Features, Substance Induced Psychotic Disorder, Schizotypal Personality Disorder and Mood Disorder with Psychotic Features.

7. The method of claim 1, wherein the neuropsychiatric disease is chronic.

8. A method of monitoring efficacy of treatment of a neuropsychiatric disease in a human subject, comprising detecting a reduction in concentration of a fragment of a neural cell adhesion molecule in cerebrospinal fluid of the subject, whereby the reduction in concentration of the fragment of the neural cell adhesion molecule in cerebrospinal fluid of the subject indicates efficacy of treatment of the neuropsychiatric disease.

9. The method of claim 8, wherein the fragment is selected from the group consisting of cN-CAM, dN-CAM, SEC N-CAM 108, SEC N-CAM 115, VASE N-CAM 155 and VASE N-CAM 165.

10. A method of screening for a compound effective in treating a neuropsychiatric disease associated with the presence of an elevated level of a fragment of a neural cell adhesion molecule in brain or cerebrospinal fluid of a human subject, comprising the following steps:
a) contacting a sample of brain cortex with the compound; and
b) detecting a reduction of breakdown of the neural cell adhesion molecule into the fragment, whereby the reduction of breakdown of the neural cell adhesion molecule into the fragment indicates that the compound is effective in treating the neuropsychiatric disease.

11. The method of claim 10, wherein the neuropsychiatric disease is selected from the group consisting of Schizophrenia, Bipolar Disorder I, Bipolar Disorder I with Psychotic Features, Bipolar Disorder II, Bipolar Disorder II with Psychotic Features, Psychotic Disorder Not Otherwise Specified, Schizophreniform Disorder, Schizoaffective Disorder, Unipolar Disorder, Unipolar Disorder with Psychotic Features, Substance Induced Psychotic Disorder, Schizotypal Personality Disorder and Mood Disorder with Psychotic Features.

12. A fragment of N-CAM, wherein the fragment is selected from the group consisting of dN-CAM, VASE N-CAM 155, VASE N-CAM 165 and VASE N-CAM 200.

13. A method of treating a neuropsychiatric disease in a human subject comprising administering a therapeutically effective amount of a compound which reduces breakdown of an endogenous protease inhibitor in brain, whereby administering the compound to the subject treats the neuropsychiatric disease.

14. The method of claim 13, wherein the compound is a protease inhibitor.

15. A method of screening for a compound effective in reducing breakdown of an endogenous protease inhibitor in human brain cortex, comprising the following steps:
a) contacting a sample of brain cortex with the compound; and
b) detecting a rise in a level or maintenance of a steady-state level of the endogenous protease inhibitor, whereby the rise in the level or maintenance of the steady-state level of the endogenous protease inhibitor indicates that the compound is effective in reducing the breakdown of the endogenous protease inhibitor in brain.

16. A composition comprising a protease inhibitor and a neuraminidase inhibitor in a pharmaceutically acceptable carrier.

17. The composition of claim 16, wherein the protease inhibitor is selected from the group consisting of aspartic protease inhibitors, serine protease inhibitors, cysteine protease inhibitors and aminopeptidase inhibitors.

18. The composition of claim 17, wherein the protease inhibitor is selected from the group consisting of Nelfinavir, Saquinavir, Indinavir, Amprenavir, Ritonavir, Aprotinin, Pepstatin, AG1776, ABT-387, Beta-secretase inhibitors, AEBSF, Leupeptin, Elastatinal, Serpins, Antipain, APMSF, PMSF, AG7088, E-64, Betastatin and Amastatin.

19. The composition of claim 16, wherein the neuraminidase inhibitor is selected from the group consisting of Zanamivir, Oseltamivir, RWJ-270201, GS 4071 and GS 4104.

20. A method of reducing breakdown of N-CAM into a fragment in a brain of a human subject, comprising administering a therapeutically effective amount of a composition comprising at least one compound selected from the group consisting of protease inhibitors and neuraminidase inhibitors, whereby administering the composition to the subject reduces the breakdown of N-CAM into the fragment in the brain of the subject.
